(19) United States
(12) Patent Application Publication
(10) Pub. No.: US 2023/0227381 A1
(43) Pub. Date: Jul. 20, 2023

(54) METHOD FOR PRODUCING PROPYLENE OLIGOMER

(71) Applicant: IDEMITSU KOSAN CO.,LTD., Tokyo (JP)

(72) Inventors: Shun FUKASAWA, Chiyoda-ku (JP); Jun SHAMOTO, Chiyoda-ku (JP); Tetsuya SARUWATARI, Chiyoda-ku (JP); Toshiki NAGAMACHI, Chiyoda-ku (JP); Shohjiroh TANASE, Chiyoda-ku (JP)

(73) Assignee: IDEMITSU KOSAN CO.,LTD., Tokyo (JP)

(21) Appl. No.: 17/769,476

(22) PCT Filed: Sep. 30, 2020
(86) PCT No.: PCT/JP2020/037293

§ 371 Date: Apr. 15, 2022

(30) Foreign Application Priority Data
Oct. 18, 2019 (JP) 2019-191258

(51) Int. Cl. C07C 2/18; C07C 5/27; B01J 27/16; B01J 29/40; B01J 35/10; B01D 3/14

(52) U.S. Cl. CPC C07C 2/18; C07C 5/2716; B01J 27/16; B01J 29/40; B01J 35/1019; B01J 35/1023; B01J 35/1038; B01J 35/1042; B01D 3/14; C07C 2529/40; C07C 2527/173

(57) ABSTRACT

Provided is a method for producing a propylene oligomer, which is advantageous in that a lowly branched propylene oligomer can be obtained at high selectivity. A method for producing a propylene oligomer, including an oligomerization step of oligomerizing propylene at lower than 160° C. in the presence of at least one member selected from a group consisting of a catalyst containing crystalline molecular sieve and a catalyst containing phosphoric acid, a fractional distillation step of obtaining a fraction containing a propylene trimer, a propylene tetramer, or a mixture thereof, and an isomerization step of isomerizing the propylene trimer, propylene tetramer, or mixture thereof contained in the fraction in the presence of a catalyst containing phosphoric acid.

### TECHNICAL FIELD

The present invention relates to a method for producing a propylene oligomer.

### BACKGROUND ART

Propylene oligomers having 9 or 12 carbon atoms (propylene trimer and propylene tetramer) obtained by subjecting propylene to polymerization at a low degree are useful as a raw material for an alcohol, a carboxylic acid, and the like, and a monomer for a polyolefin.

Particularly, a propylene trimer is widely used as a raw material for mercaptan, and the like. Further, a propylene tetramer is used as a raw material for a detergent and a plasticizer, and the like. With respect to these raw materials, an oligomer which is branched at a low degree (lowly branched oligomer) is particularly useful.

Conventionally, a propylene oligomer has been produced using a catalyst containing phosphoric acid, such as a solid phosphoric acid catalyst, but recently, studies are being made on the production of a propylene oligomer using zeolite as a catalyst. The solid phosphoric acid catalyst has poor mechanical strength, and hence has a short life of the catalyst, and, for stably obtaining a propylene oligomer using the catalyst for a long period of time, the catalyst must be replaced with high frequency. Attempts are made to extend the life of the catalyst for removing such disadvantages.

For example, as a method for improving the catalyst life by suppressing heat generation without using a diluent, PTL 1 discloses an oligomerization method for an olefin hydrocarbon, in which an olefin hydrocarbon is successively contacted with a crystalline molecular sieve catalyst and a solid phosphoric acid catalyst.

Further, oligomerization of an olefin using a plurality of catalysts is studied.

For example, PTL 2 discloses an oligomerization or polymerization apparatus having fixed beds being capable of independently controlling the temperature and having different catalysts.

### CITATION LIST

### Patent Literature

PTL 1: WO 2005/118513

PTL 2: WO 2007/024330

### SUMMARY OF INVENTION

### Technical Problem

When a molecular sieve catalyst (zeolite catalyst) having a relatively long life is used, the catalyst life can be extended, but it is likely that the obtained propylene oligomer has a structure different from the intended structure, making it difficult to obtain a lowly branched oligomer useful as a raw material for a lubricant and a detergent.

Meanwhile, even when two types of catalysts, for example, a molecular sieve catalyst and a solid phosphoric acid catalyst are used in combination as described in the above-mentioned PTLs 1 and 2, for obtaining an oligomer having the intended structure, there is a need to conduct a reaction satisfactorily using the solid phosphoric acid catalyst, making it difficult to prevent degradation of the catalyst.

Further, when a reaction is conducted under high temperature conditions and the like for obtaining an oligomer having the intended structure, it is difficult to control the reaction, so that a modification product is caused, or an oligomer having a required molecular weight cannot be obtained, lowering the selectivity.

Therefore, there have been desired a method for efficiently obtaining, at high selectivity, a lowly branched propylene oligomer useful as a raw material for a lubricant and a detergent as well as a method for obtaining a propylene oligomer while preventing degradation of the catalyst and extending the catalyst life.

Accordingly, a task of the present disclosure is to provide a technique that relates to a method for producing a propylene oligomer, which is advantageous in that a lowly branched propylene oligomer can be efficiently obtained at high selectivity. Further, another task of the present disclosure is to provide a technique that relates to a method for producing a propylene oligomer, which is advantageous in that a lowly branched propylene oligomer can be efficiently obtained at high selectivity while extending the catalyst life.

Further, recently, chemical products, such as a surfactant, an oily material, a solvent, and a polymer, are required to have various functions, such as detergency, compatibility, and blending stability, and a propylene oligomer which is a raw material for these chemical products is needed to have a higher degree of branching. For example, when a propylene oligomer having an alkyl portion which is branched at a high degree (highly branched) is used in a surfactant or the like, it is expected that the surfactant has poor crystalline properties and hence has improved compatibility with various oils, and thus is improved particularly in the detergency at low temperatures. Further, when used in a solvent, the resultant solvent can be expected to exhibit high dissolving power.

However, when using a conventional solid phosphoric acid catalyst, it is difficult to obtain a highly branched propylene oligomer at a high concentration.

Accordingly, a task of the present disclosure is to provide a technique that relates to a propylene oligomer containing a highly branched propylene tetramer at a high concentration, and a method for producing, at a high concentration, a propylene oligomer containing a highly branched propylene tetramer at a high concentration.

### Solution to Problem

The present inventors have conducted extensive and intensive studies with a view toward solving the above-mentioned problems. As a result, it has been found that the above-mentioned problems can be solved by using a method in which propylene is oligomerized in the presence of a catalyst at a specific temperature, and subjected to fractional distillation and isomerized in the presence of a catalyst containing phosphoric acid, and the invention has been completed.

Specifically, according to an embodiment of the present disclosure, there can be provided a technique that relates to a method for producing a propylene oligomer, which includes an oligomerization step of oligomerizing propylene at lower than 160° C. in the presence of at least one member selected from a group consisting of a catalyst containing crystalline molecular sieve and a catalyst containing phosphoric acid, a fractional distillation step of obtaining a fraction containing a propylene trimer, a propylene tetramer, or a mixture thereof, and an isomerization step of isomerizing the propylene trimer, the propylene tetramer, or the mixture thereof contained in the fraction in the presence of a catalyst containing phosphoric acid.

Further, the present inventors have conducted extensive and intensive studies with a view toward solving the above-mentioned problems. As a result, it has been found that the above-mentioned problems can be solved by using a method in which an oligomer containing a propylene trimer, a propylene tetramer, or a mixture thereof is isomerized in the presence of a catalyst at a specific pressure, and the invention has been completed.

Specifically, according to an embodiment of the present disclosure, there can be provided a technique that relates to a method for producing a propylene oligomer, which includes a step of isomerizing an oligomer containing a propylene trimer, a propylene tetramer, or a mixture thereof at less than the critical pressure of propylene in the presence of at least one member selected from a group consisting of a catalyst containing crystalline molecular sieve and a catalyst containing phosphoric acid.

Furthermore, the present inventors have conducted extensive and intensive studies with a view toward solving the above-mentioned problems. As a result, it has been found that, by using a zeolite catalyst containing a large amount of micropores, specific oligomerization proceeds, so that a highly branched propylene tetramer having a specific structure is produced at a high concentration, and the invention has been completed.

Specifically, an embodiment of the present disclosure is a propylene oligomer containing a propylene tetramer having a 4,6,6-trimethyl-3-nonene concentration of 30% by mass or more. Further, according to another embodiment of the present disclosure, there can be provided a technique that relates to a method for producing a propylene oligomer, which includes a step of oligomerizing propylene in the presence of a catalyst containing crystalline molecular sieve, wherein when a BET specific surface area of the crystalline molecular sieve, as obtained by a nitrogen adsorption method, is taken as “a” [m²/g] and a micropore specific surface area of the crystalline molecular sieve, as obtained by subjecting an adsorption isotherm measured by a nitrogen adsorption method to analysis in accordance with a t-plot method, is taken as “b” [m²/g], a/b is 1.8 or less.

### Advantageous Effects of Invention

According to an embodiment of the present disclosure, there can be provided a technique that relates to a method for producing a propylene oligomer, which is advantageous in that a lowly branched propylene oligomer can be efficiently obtained while extending the catalyst life. Further, according to another embodiment of the present disclosure, there can be provided a technique that relates to a method for producing a propylene oligomer, which is advantageous in that a lowly branched propylene oligomer can be efficiently obtained.

Furthermore, according to still another embodiment of the present disclosure, a propylene oligomer containing a highly branched propylene tetramer having a specific structure at a high concentration can be obtained. Further, according to still another embodiment of the present disclosure, there can be provided a technique that relates to a method for producing a propylene oligomer containing a highly branched propylene tetramer having a specific structure at a high concentration.

### BRIEF DESCRIPTION OF DRAWINGS

FIG. 1 is a GC chart of the propylene oligomer having 12 carbon atoms obtained by oligomerization conducted in the presence of a solid phosphoric acid catalyst.

FIG. 2 is a GC chart of the propylene oligomer having 12 carbon atoms obtained by oligomerization conducted in the presence of a crystalline molecular sieve in which the ratio of the BET specific surface area to the micropore specific surface area (a/b) is more than 1.8.

FIG. 3 is a GC chart of the propylene oligomer having 12 carbon atoms obtained by oligomerization conducted in the presence of a crystalline molecular sieve in which the ratio of the BET specific surface area to the micropore specific surface area (a/b) is 1.8 or less.

### DESCRIPTION OF EMBODIMENTS

In the present specification, the term “to” used in connection with the expression of a range of values means that the respective values shown before and after “to” are the lower limit value and the upper limit value of the range.

### First Embodiment

The first embodiment of the present disclosure is a technique that relates to a method for producing a propylene oligomer, which includes the oligomerization step of oligomerizing propylene at lower than 160° C. in the presence of at least one member selected from the group consisting of a catalyst containing crystalline molecular sieve and a catalyst containing phosphoric acid, the fractional distillation step of obtaining a fraction containing a propylene trimer, a propylene tetramer, or a mixture thereof, and the isomerization step of isomerizing the propylene trimer, propylene tetramer, or mixture thereof contained in the fraction in the presence of a catalyst containing phosphoric acid.

Hereinbelow, the first embodiment is described in detail.

### [Method for Producing a Propylene Oligomer]

The method for producing a propylene oligomer of the first embodiment includes the oligomerization step of oligomerizing propylene at lower than 160° C. in the presence of at least one member selected from the group consisting of a catalyst containing crystalline molecular sieve and a catalyst containing phosphoric acid, the fractional distillation step of obtaining a fraction containing a propylene trimer, a propylene tetramer, or a mixture thereof, and the isomerization step of isomerizing the propylene trimer, propylene tetramer, or mixture thereof contained in the fraction in the presence of a catalyst containing phosphoric acid.

The reason that, by the production method of the first embodiment, a lowly branched propylene oligomer can be obtained at high selectivity while extending the catalyst life is not clear, but is presumed as follows.

It is considered that, by conducting the oligomerization step using the above-mentioned catalyst at a temperature as low as less than 160° C., the intended trimer and tetramer can be obtained while preventing an unnecessary side reaction and degradation of the catalyst. Particularly, when using a catalyst containing phosphoric acid, for maintaining the activity, it is necessary to introduce water into the reaction system, and, when the reaction temperature is high, the amount of the water introduced must be increased. In the production method of the first embodiment, it is considered that the reaction conducted at a low temperature enables reduction of the amount of the water introduced, making it possible to suppress a lowering of the mechanical strength of the catalyst.

The obtained polymer is then subjected to fractional distillation and isomerization, and it is considered that, by subjecting the oligomer containing a trimer and a tetramer as a main component obtained by the reaction to isomerization reaction using a catalyst containing phosphoric acid, an oligomer having a low degree of branching and the intended polymerization degree can be obtained at high selectivity. Further, it is considered that, in the isomerization step, a polymerization reaction of the residual propylene or a light olefin, such as a dimer, does not occur, and thus the heat of reaction can be reduced, making it possible to suppress degradation of the catalyst. Furthermore, it is considered that the oligomer containing a trimer and a tetramer as a main component is used in the reaction, and hence the isomerization reaction can be performed in a small scale, so that a lowly branched propylene oligomer can be efficiently obtained.

### <Oligomerization Step>

The present step is the step of oligomerizing propylene at lower than 160° C. in the presence of at least one member selected from the group consisting of a catalyst containing crystalline molecular sieve and a catalyst containing phosphoric acid.

A polymerization method in which a lower olefin, representatively propylene is contacted with a solid acid catalyst to obtain an oligomer of the olefin is called cationic polymerization. The oligomer product obtained by cationic polymerization is generally in the form of a mixture of an olefin dimer, trimer, tetramer, and higher oligomers. Further, each oligomer is produced through a complicated reaction mechanism, and therefore is rarely obtained as an olefin having a single carbon skeleton and a single position of double bond, and is generally obtained in the form of a mixture of various isomers.

In the present step, using a catalyst containing crystalline molecular sieve or a catalyst containing phosphoric acid, cationic polymerization is conducted at a relatively low temperature, and hence, while preventing degradation of the catalyst, a propylene trimer and a propylene tetramer useful as various raw materials are obtained.

It is preferred that the crystalline molecular sieve contained in the catalyst used in the present step is zeolite.

Examples of the crystalline molecular sieves include 10-membered ring zeolite and 12-membered ring zeolite, and preferred is at least one member selected from the group consisting of 10-membered ring zeolite and 12-membered ring zeolite, and more preferred is 10-membered ring zeolite.

Examples of the 10-membered ring zeolite include those of an MFI type (another name: ZSM-5), an MFS type (another name: ZSM-57), a TON type (another name: ZSM-22), an MTT type (another name: ZSM-23), an MEL type (another name: ZSM-11), an FER type, an MRE type (another name: ZSM-48), and an MWW type (another name: MCM-22), and those of an MFI type, an MFS type, and an MTT type are preferred, and 10-membered ring zeolite of an MFI type is more preferred. That is, as the crystalline molecular sieve, MFI-type zeolite is more preferred.

From the viewpoint of improving the activity, the total surface area (BET specific surface area for the all surface) of the 10-membered ring zeolite as measured by a nitrogen adsorption method is preferably 200 m²/g or more, more preferably 300 m²/g or more, further preferably 400 m²/g or more.

From the viewpoint of allowing the reaction to more efficiently proceed, the ratio of the outer surface area (specific surface area of pores other than micropores as obtained by a t-plot method) to the total surface area of the 10-membered ring zeolite, as measured by a nitrogen adsorption method, (outer surface area/total surface area) is preferably 0.4 or more, more preferably 0.5 or more, further preferably 0.6 or more. The term “BET specific surface area” means a specific surface area determined by a BET analysis using an adsorption isotherm measured by a nitrogen adsorption method. The term “specific surface area of pores other than micropores” means a specific surface area obtained by subjecting an adsorption isotherm measured by a nitrogen adsorption method to analysis in accordance with a t-plot method.

From the viewpoint of allowing the reaction to more efficiently proceed, the crystal diameter of the 10-membered ring zeolite as observed by a SEM (scanning electron microscope) is preferably 1 μm or less, more preferably 0.5 μm or less, further preferably 0.1 μm or less.

From the viewpoint of allowing the reaction to efficiently proceed, the silicon/aluminum molar ratio (Si/Al) of the 10-membered ring zeolite is preferably 100 or less, more preferably 50 or less, further preferably 25 or less.

From the viewpoint of allowing the reaction to efficiently proceed, the acid amount of the 10-membered ring zeolite as measured by NH₃-TPD is preferably 150 μmol/g or more, more preferably 200 μmol/g or more, further preferably 250 μmol/g or more.

For improving the moldability for catalyst, a binder may be used when molding zeolite. As the binder, a metal oxide, such as alumina, silica, or clay, can be used, and, from the viewpoint of the effect on the mechanical strength, cost, and acid site and the like, the binder is preferably alumina. As the amount of the binder used is reduced, the amount of the zeolite which is an active site is increased, and therefore the amount of the binder is preferably 50% by mass or less, more preferably 30% by mass or less, further preferably 20% by mass or less.

Examples of the 12-membered ring zeolite include those of an FAU type (another name: Y-type zeolite), a BEA type (another name: 13 zeolite), an MOR type, an MTW type (another name: ZSM-12), an OFF type, and an LTL type (another name: L-type zeolite), and those of an FAU type and a BEA type are preferred, and 12-membered ring zeolite of a BEA type is more preferred.

From the viewpoint of improving the activity, the total surface area (BET specific surface area for the all surface) of the 12-membered ring zeolite as measured by a nitrogen adsorption method is preferably 200 m²/g or more, more preferably 300 m²/g or more, further preferably 400 m²/g or more.

From the viewpoint of allowing the reaction to more efficiently proceed, the ratio of the outer surface area (specific surface area of pores other than micropores as obtained by a t-plot method) to the total surface area of the 12-membered ring zeolite, as measured by a nitrogen adsorption method, (outer surface area/total surface area) is preferably 0.4 or more, more preferably 0.5 or more, further preferably 0.6 or more.

From the viewpoint of allowing the reaction to more efficiently proceed, the crystal diameter of the 12-membered ring zeolite as observed by a SEM is preferably 1 μm or less, more preferably 0.5 μm or less, further preferably 0.1 μm or less. From the viewpoint of allowing the reaction to efficiently proceed, the silicon/aluminum molar ratio (Si/A1) of the 12-membered ring zeolite is preferably 100 or less, more preferably 50 or less, further preferably 25 or less.

From the viewpoint of allowing the reaction to efficiently proceed, the acid amount of the 12-membered ring zeolite as measured by NH₃-TPD is preferably 150 μmol/g or more, more preferably 200 μmol/g or more, further preferably 250 μmol/g or more.

For improving the moldability for catalyst, a binder may be used when molding zeolite. As the binder, a metal oxide, such as alumina, silica, or clay mineral, can be used, and, from the viewpoint of the effect on the mechanical strength, cost, and acid site and the like, the binder is preferably alumina. As the amount of the binder used is reduced, the amount of the zeolite which is an active site is increased, and therefore the amount of the binder is preferably 50% by mass or less, more preferably 30% by mass or less, further preferably 20% by mass or less.

It is preferred that the catalyst containing crystalline molecular sieve is used as a fixed bed catalyst in such a way that a fixed bed reactor is filled with the catalyst.

The catalyst containing phosphoric acid used in the present step is preferably a solid phosphoric acid catalyst.

The solid phosphoric acid catalyst is a catalyst having phosphoric acid supported on a carrier.

Examples of phosphoric acids include orthophosphoric acid, pyrophosphoric acid, and triphosphoric acid, and orthophosphoric acid is preferred. The solid phosphoric acid catalyst contains free phosphoric acid preferably in an amount of 16% by mass or more, and, for improving the catalytic activity, more preferably in a larger amount of free phosphoric acid. Generally, the solid phosphoric acid catalyst contains free phosphoric acid in an amount of 16 to 20% by mass.

Examples of carriers include diatomaceous earth, kaolin, and silica, and diatomaceous earth is preferred.

The carrier may contain an additive for improving the strength of the catalyst. Examples of additives include talc, clay mineral, and iron compounds, such as iron oxide.

The solid phosphoric acid catalyst can be obtained as follows.

It is preferred that phosphoric acid and a carrier are first mixed with each other to obtain a material in a paste form or in a clay form, and the obtained material is formed into a pellet form or a particle form. After the subsequent drying and calcination, the resultant material may be crushed into a particle form.

Then, the material in a paste form or in a clay form is dried, and then calcined to obtain catalyst pellets or catalyst particles.

The temperature for drying is preferably 100 to 300° C., more preferably 150 to 250° C.

The temperature for calcination is preferably 300 to 600° C., more preferably 350 to 500° C.

The catalyst containing phosphoric acid preferably contains water. As examples of methods for causing the catalyst containing phosphoric acid to contain water, there can be mentioned a method in which water vapor is allowed to flow through the above-mentioned catalyst pellets or catalyst particles to cause the catalyst to contain water, and a method in which the catalyst containing phosphoric acid and water are added to the reactor.

The content of phosphoric acid, in terms of phosphoric acid anhydride (P₂O₅), in the solid phosphoric acid catalyst is preferably 30 to 60% by mass, more preferably 40 to 50% by mass.

The content of the carrier in the solid phosphoric acid catalyst is preferably 40 to 80% by mass, more preferably 50 to 60% by mass.

It is preferred that the catalyst containing phosphoric acid is used as a fixed bed catalyst in such a way that a fixed bed reactor is filled with the catalyst.

In the present step, it is preferred that, before initiating the reaction, a pretreatment for removing impurities from the catalyst is conducted. As a method for pretreatment, preferred is a method in which an inert gas, such as nitrogen or LPG, is increased in temperature and a flow of the gas at a high temperature is passed through the reactor.

The temperature for pretreatment is preferably 100 to 500° C., more preferably 150 to 400° C., further preferably 150 to 300° C. The time for pretreatment varies depending on the size of the reactor, but is preferably 1 to 20 hours, more preferably 2 to 10 hours.

Further, it is preferred that, before initiating the reaction, the water content of the catalyst is controlled. In the case of the catalyst containing crystalline molecular sieve, for improving the catalytic activity, it is preferred to remove water, and, for increasing the life of the catalyst, it is preferred to add water. As a method for removing water, the above-mentioned pretreatment method is preferably used. In the case of the catalyst containing phosphoric acid, for improving the catalytic activity, it is preferred to introduce water.

Then, propylene is introduced.

The propylene introduced may be used in the form of a mixture with a gas which is inert with respect to the present reaction, but, in the present step of oligomerizing propylene, the concentration of propylene in the reaction mixture except the catalyst is preferably 55% by volume or more, more preferably 60% by volume or more, further preferably 65% by volume or more, still further preferably 70% by volume or more.

The reaction temperature in the present step of oligomerizing propylene is lower than 160° C., preferably 90° C. or higher and lower than 160° C., more preferably 120° C. or higher and lower than 160° C., further preferably 140° C. or higher and 155° C. or lower. When a catalyst containing phosphoric acid is used as a catalyst, the reaction temperature is preferably 130° C. or higher and lower than 160° C., more preferably 140° C. or higher and lower than 160° C., further preferably 140° C. or higher and 155° C. or lower, and, when a catalyst containing crystalline molecular sieve is used as a catalyst, the reaction temperature is preferably 90° C. or higher and lower than 160° C., more preferably 120° C. or higher and lower than 160° C., further preferably 140° C. or higher and 155° C. or lower. By conducting the reaction at lower than 160° C., the propylene oligomer can be obtained in high yield while suppressing degradation of the catalyst.

The reaction temperature is an average temperature in the reactor, which indicates an average of the temperature of the upstream side and the temperature of the downstream side of the portions of the reactor in contact with the catalyst.

The liquid hourly space velocity in the present step of oligomerizing propylene is preferably 5 hour⁻¹ (h⁻¹) or less, more preferably 4 h⁻¹ or less, further preferably 3 h⁻¹ or less, still further preferably 2 h⁻¹ or less. When the liquid hourly space velocity is 5 h⁻¹ or less, the propylene trimer, propylene tetramer, or mixture thereof can be obtained in high yield.

The preliminary reaction time in the present step of oligomerizing propylene is preferably 100 hours or more, preferably 200 hours or more, preferably 250 hours or more, preferably 270 hours or more. By providing the preliminary reaction time before obtaining the reaction product, the catalyst can be stabilized, so that the propylene trimer, propylene tetramer, or mixture thereof can be obtained in high yield.

The propylene conversion in the present step is preferably 50 to 99.9%, more preferably 50 to 99%, further preferably 60 to 97%, still further preferably 70 to 95%.

In the present step, for the purpose of removing the heat from the reactor or reducing the amount of the unreacted propylene, recycling can be made by permitting the unreacted propylene and light oligomers produced by the reaction, which are discharged from the outlet of the reactor, to go back to the reactor. Light oligomers include, for example, a dimer of propylene. When recycling is conducted, from the viewpoint of the production efficiency, the ratio of the fresh feed (propylene as a raw material) and the recycle (the unreacted propylene and light oligomers) (R/F) is preferably 0.1 to 10, more preferably 0.3 to 6, further preferably 1 to 3.

### <Fractional Distillation Step>

The method for producing a propylene oligomer of the first embodiment includes the fractional distillation step of obtaining a fraction containing a propylene trimer, a propylene tetramer, or a mixture thereof.

The present fractional distillation step is preferably conducted for the following purposes.

(1) Removal of impurities: which is conducted for removing low molecular-weight compounds (for example, a propylene dimer) and high molecular-weight compounds (a pentamer and polymers), which are by-products produced by oligomerization, modification products produced due to a side reaction, such as cracking, e.g., an olefin having carbon atoms, the number of which is not a multiple of 3, and the like.
(2) Separation of the component used in the isomerization step: which is conducted for obtaining a propylene trimer, a propylene tetramer, or a mixture thereof at a high concentration.

Fractional distillation may be simultaneously performed for the both purposes (1) and (2) above, and fractional distillation is performed for the purpose (1) and then fractional distillation may be performed for the purpose (2). Especially, it is preferred that fractional distillation is performed for the purpose (1) and then fractional distillation is performed for the purpose (2).

The conditions for fractional distillation particularly for the purpose (2) are described below.

By conducting the present fractional distillation step, the component to be used in the isomerization step can be efficiently obtained. When the isomerization step is performed just after the oligomerization step without conducting the present fractional distillation step, not only the needed oligomer but also low molecular-weight compounds, modification products and the like are introduced into the reactor at the same time, and thus a side reaction of these compounds, such as cracking, disadvantageously proceeds, so that the yield of the intended isomer of the propylene trimer, propylene tetramer, or mixture thereof is reduced. In addition, the residual propylene and light olefins produced in the oligomerization step, such as a propylene dimer, undergo polymerization also in the isomerization step, and therefore heat generation caused due to the polymerization reaction increases the reaction temperature. For this reason, in the isomerization step, the reactor having an increased size is inevitably used, and the burden on fractionation and purification after the isomerization step is considerably increased, and such a problem is disadvantageous from the viewpoint of the energy in the isomerization step and the cost.

Further, by conducting the present fractional distillation step, the resultant fraction contains no propylene and light olefin, and therefore the reaction pressure in the subsequent isomerization step at a high temperature can be reduced, making it possible to suppress the installation cost for the reactor.

In the present fractional distillation step, a fraction containing a mixture of a propylene trimer and a propylene tetramer as a main component is obtained, and may be subjected to fractionation after the isomerization reaction, or a propylene trimer or a propylene tetramer and the needed oligomer may be selected and separated and subjected to the isomerization step. Especially, it is preferred that a fraction containing a mixture of a propylene trimer and a propylene tetramer as a main component is obtained and subjected to fractionation after the isomerization reaction. By obtaining a fraction containing a propylene trimer, a propylene tetramer, or a mixture thereof as a main component in the present step as mentioned above, not only can the size of the reactor used in the isomerization step be further reduced, but also the needed isomer can be obtained in high yield, and the fractionation and purification after the isomerization step can be more easily conducted.

Conditions for the fractional distillation vary depending on the pressure, the size of the distillation apparatus, the number of plates of the distillation column, and the like, and further vary depending on the production efficiency, the intended purity, and the use, but it is preferred that the fractional distillation is conducted under conditions such that an olefin having 9 carbon atoms or having 12 carbon atoms which is a propylene trimer or propylene tetramer is obtained.

When an olefin having 9 carbon atoms which is a propylene trimer is mainly obtained, the distilling set temperature for the distillation under atmospheric pressure (1 atm) is preferably 120 to 160° C., more preferably 125 to 155° C., further preferably 130 to 150° C., still further preferably 130 to 145° C.

When an olefin having 12 carbon atoms which is a propylene tetramer is mainly obtained, the distilling set temperature for the distillation under atmospheric pressure (1 atm) is preferably 150 to 230° C., more preferably 160 to 220° C., further preferably 170 to 210° C.

Further, when a mixture of the propylene trimer and propylene tetramer is mainly obtained, the distilling set temperature for the distillation under atmospheric pressure (1 atm) is preferably 120° C. or higher, more preferably 125° C. or higher, further preferably 130° C. or higher. The upper limit of the temperature varies depending on the amount of the higher molecular-weight polymer produced, but, when the amount of the higher molecular-weight polymer produced is small, distillation may be performed until all the remaining material is distilled. When the amount of the higher molecular-weight polymer is large, the upper limit of the temperature is preferably 230° C. or lower, more preferably 220° C. or lower, further preferably 210° C. or lower.

### <Isomerization Step>

The present step is the step of isomerizing the propylene trimer, propylene tetramer, or mixture thereof contained in the above-mentioned fraction in the presence of a catalyst containing phosphoric acid.

With respect to the catalyst containing phosphoric acid used in the present step, the same catalyst as used in the <Oligomerization step> above can be used, and preferred catalysts are similar to those mentioned above.

By using the catalyst containing phosphoric acid, the intended lowly branched propylene oligomer can be efficiently obtained at high selectivity.

In the present step, it is preferred that, before initiating the reaction, the water content of the catalyst is controlled. For improving the catalytic activity, it is desired to introduce water.

The present isomerization step is preferably conducted at 160° C. or higher. The reaction temperature in the present step is preferably 160° C. or higher, preferably 160 to 260° C., more preferably 160 to 230° C., further preferably 170 to 220° C., still further preferably 180 to 200° C. By conducting the reaction at 160° C. or higher, the intended propylene oligomer having a low degree of branching can be efficiently obtained in high yield.

The reaction temperature is an average temperature in the reactor, which indicates an average of the temperature of the upstream side and the temperature of the downstream side of the portions of the reactor in contact with the catalyst.

The reaction pressure in the present isomerization step is preferably less than the critical pressure of propylene. The expression “critical pressure of propylene” means a pressure at the critical point of propylene, specifically 4.66 MPa (absolute pressure). By virtue of the above-mentioned fractional distillation step, the resultant fraction contains no propylene and light olefin. For this reason, the propylene trimer and propylene tetramer, which are a main constituent of the isomerization raw material, can keep in a liquid phase at the above-mentioned reaction temperature without a need of increasing the pressure to the critical pressure of propylene or more. By conducting the isomerization in a liquid phase, it is possible to improve the reaction efficiency. The reaction pressure in the isomerization step is preferably 3.00 MPa or less, more preferably 2.00 MPa or less, further preferably 1.50 MPa or less, especially preferably 1.00 MPa or less. The reaction pressure is indicated in terms of a gauge pressure. Further, from the viewpoint of the pressure at which the propylene trimer that is a main raw material keeps in a liquid phase, the reaction pressure in the isomerization step is preferably 0.00 MPa or more (atmospheric pressure or more), more preferably 0.05 MPa or more. The reaction pressure is indicated in terms of a gauge pressure.

The liquid hourly space velocity in the present isomerization step is preferably 0.1 to 10 h⁻¹, more preferably 0.2 to 8 h⁻¹, further preferably 0.5 to 6 h⁻¹, still further preferably 1 to 4 h⁻¹. When the liquid hourly space velocity is in the above-mentioned range, the intended propylene oligomer having a low degree of branching can be obtained without markedly lowering the yield of the propylene trimer and tetramer.

By conducting the present isomerization step, the propylene oligomer having the intended polymerization degree can be obtained at high selectivity.

The by-product selectivity in the present isomerization step is preferably 25% by mass or less, more preferably 15% by mass or less. By-products are compounds other than the propylene trimer and tetramer which are a product, and the propylene dimer which can be used for producing a product by further conducting the oligomerization step through recycling or the like, specifically, high molecular-weight compounds (propylene pentamer and polymers) produced by a polymerization reaction, modification products produced due to a side reaction, such as cracking, e.g., an olefin having carbon atoms, the number of which is not a multiple of 3, and the like. The by-product selectivity is a content of by-products in the reaction mixture obtained after the isomerization step.

The method for producing a propylene oligomer of the first embodiment may include the fractionation step after the present isomerization step. By subjecting the obtained isomer to fractionation, it is possible to remove impurities and modification products from the isomer.

The distillation conditions in the fractionation step which is conducted after the present isomerization step vary depending on the intended oligomer, but are preferably the conditions described above in connection with <Fractional distillation step>.

### <Propylene Oligomer Obtained by the Production Method>

It is preferred that the propylene oligomer obtained by the production method of the first embodiment has a low degree of branching and has a small Type V olefin content.

An explanation is made on “Type V olefin” and the olefin type of the propylene oligomer.

The olefin type of the propylene oligomer can be classified according to the degree of substitution of the double bond and the position thereof as shown in Table 1. In the formulae, C represents a carbon atom, H represents a hydrogen atom, and =represents a double bond. Further, in the formulae, R represents an alkyl group, and R's may be the same or different, and, in the propylene trimer, the total number of carbon atoms of R per molecule is 7, and, in the propylene tetramer, the total number of carbon atoms of R per molecule is 10.

That is, an olefin type of a propylene oligomer having a structure of RRC=CRR is referred to as “Type V olefin”.

Type I is frequently called “vinyl type”, and Type III is frequently called “vinylidene type”.

TABLE 1

| Olefin type | Structural formula | Degree of substitution |
|---|---|---|
| Type I | RHC = CH₂ | 1-Substitution (Vinyl type) |
| Type II | RHC = CHR | 2-Substitution |
| Type III | RRC = CH₂ | 2-Substitution (Vinylidene type) |
| Type IV | RRC = CHR | 3-Substitution |
| Type V | RRC = CRR | 4-Substitution |

Oligomer isomers having different degrees of branching or positions of double bond likely have different reactivities in the downstream process using the oligomer as a feed raw material. For example, an isomer having a low degree of branching is highly active in a reaction, such as a hydroformylation reaction (oxo process). The reactivity of an isomer is considered to vary depending on the steric environment surrounding the double bond of the isomer.

Further, the different degrees of branching or positions of double bond of oligomer isomers likely affect not only the reactivity of the oligomer isomer but also the product properties in the downstream process using the oligomer as a feed raw material. An oligomer containing a large amount of a linear or lowly branched isomer, such as the propylene oligomer obtained by the production method of the first embodiment, is useful as a raw material for a lubricant and a detergent.

When the propylene oligomer obtained by the production method of the first embodiment is a propylene trimer, the propylene trimer preferably has a Type V olefin concentration of 22% by mass or less, more preferably 21% by mass or less, further preferably 20% by mass or less, still further preferably 19% by mass or less, still further preferably 18% by mass or less. The lower limit of the concentration is not limited, but, from the viewpoint of the production efficiency, the lower limit is preferably 10% by mass or more, more preferably 15% by mass or more.

The Type V olefin concentration means a content (% by mass) of the Type V olefin in the propylene trimer, and, with respect to the method for measuring and determining the concentration, the method described in the Examples below is used.

When having a Type V olefin concentration of 23% by mass or less, the propylene trimer can be advantageously used as a raw material for olefin derivatives.

The propylene trimer can contain a Type IV olefin, a Type III olefin, a Type II olefin, and a Type I olefin as well as the Type V olefin.

The Type IV olefin concentration of the propylene trimer in the first embodiment is preferably 50% by mass or more, more preferably 52% by mass or more, further preferably 55% by mass or more. The upper limit of the concentration is not limited, but, from the viewpoint of the production efficiency, the upper limit is preferably 70% by mass or less, more preferably 65% by mass or less.

The Type IV olefin concentration means a content (% by mass) of the Type IV olefin in the propylene trimer, and, with respect to the method for measuring and determining the concentration, the method described in the Examples below is used.

The Type II olefin concentration of the propylene trimer in the first embodiment is preferably 14% by mass or more, preferably 15% by mass or more, more preferably 16% by mass or more, further preferably 18% by mass or more. The upper limit of the concentration is not limited, but, from the viewpoint of the production efficiency, the upper limit is preferably 25% by mass or less, more preferably 22% by mass or less.

The Type II olefin concentration means a content (% by mass) of the Type II olefin in the propylene trimer, and, with respect to the method for measuring and determining the concentration, the method described in the Examples below is used.

The propylene trimer in the first embodiment preferably has a distilling temperature (initial boiling point to end point) of 120 to 160° C., more preferably 125 to 155° C., further preferably 130 to 150° C., still further preferably 130 to 148° C., still further preferably 130 to 145° C., as measured by an atmospheric distillation test method prescribed by JIS K2254:2018. The atmospheric distillation test method is a test method in which a sample is divided into predetermined groups according to the properties, and 100 mL of a sample is subjected to distillation under the conditions to measure an initial boiling point, a distilling temperature, a distillate amount, an end point and the like.

The propylene trimer in the first embodiment preferably has a 50% by volume distilling temperature of 132 to 142° C., more preferably 134 to 140° C., further preferably 135 to 138° C., as measured by an atmospheric distillation test method prescribed by JIS

When the boiling point (distilling temperature measured by a distillation test) of the propylene trimer is in the above range, the propylene trimer can be advantageously used as a raw material for olefin derivatives as intended.

When the propylene oligomer obtained by the production method of the first embodiment is a propylene tetramer, the propylene tetramer preferably has a Type V olefin concentration of 30% by mass or less, more preferably 26% by mass or less, further preferably 22% by mass or less, still further preferably 20% by mass or less, still further preferably 18% by mass or less. The lower limit of the concentration is not limited, but, from the viewpoint of the production efficiency, the lower limit is preferably 5% by mass or more, more preferably 10% by mass or more.

The Type V olefin concentration means a content (% by mass) of the Type V olefin in the propylene trimer, and, with respect to the method for measuring and determining the concentration, the method described in the Examples below is used.

When having a Type V olefin concentration of 30% by mass or less, the propylene tetramer can be advantageously used as a raw material for olefin derivatives.

The propylene tetramer can contain a Type IV olefin, a Type III olefin, a Type II olefin, and a Type I olefin as well as the Type V olefin.

The Type IV olefin concentration of the propylene tetramer in the first embodiment is preferably 55% by mass or more, more preferably 60% by mass or more, further preferably 63% by mass or more, still further preferably 65% by mass or more. The upper limit of the concentration is not limited, but, from the viewpoint of the production efficiency, the upper limit is preferably 85% by mass or less, more preferably 75% by mass or less.

The Type IV olefin concentration means a content (% by mass) of the Type IV olefin in the propylene tetramer, and, with respect to the method for measuring and determining the concentration, the method described in the Examples below is used.

The propylene tetramer in the first embodiment preferably has a distilling temperature (initial boiling point to end point) of 150 to 230° C., more preferably 155 to 225° C., further preferably 160 to 220° C., still further preferably 165 to 215° C., still further preferably 170 to 210° C., as measured by an atmospheric distillation test method prescribed by JIS K2254:2018.

The propylene tetramer in the first embodiment preferably has a 50% by volume distilling temperature of 175 to 195° C., more preferably 180 to 190° C., further preferably 185 to 190° C., as measured by an atmospheric distillation test method prescribed by JIS K2254:2018.

When the boiling point (distilling temperature measured by a distillation test) of the propylene tetramer is in the above range, the propylene tetramer can be advantageously used as a raw material for olefin derivatives as intended.

### Second Embodiment

The second embodiment of the present disclosure is a technique that relates to a method for producing a propylene oligomer, which includes the step of isomerizing an oligomer containing a propylene trimer, a propylene tetramer, or a mixture thereof at less than the critical pressure of propylene in the presence of at least one member selected from the group consisting of a catalyst containing phosphoric acid.

When the oligomer containing a propylene trimer, a propylene tetramer, or a mixture thereof as a main component is isomerized, the isomerization reaction can be performed in a small scale, so that an oligomer having a low degree of branching and the intended polymerization degree can be obtained at high selectivity. Further, the oligomer containing a propylene trimer, a propylene tetramer, or a mixture thereof as a main component is present in a liquid phase even at a reaction pressure which is less than the critical pressure of propylene. Therefore, the method for producing a propylene oligomer of the second embodiment can improve the reaction efficiency, as compared to a method using a gas phase reaction. Further, the reaction in a liquid phase enables heavy compounds produced during the reaction to be washed away, and therefore an effect is obtained such that the life of the catalyst used in the isomerization reaction can be extended, as compared to that in a method using a gas phase reaction. Furthermore, in the method for producing a propylene oligomer of the second embodiment, a reaction can be conducted at a low pressure, and thus a high pressure-resistant reaction vessel is not needed, making it possible to reduce the production cost.

Hereinbelow, the second embodiment is described in detail.

### [Method for Producing a Propylene Oligomer]

In the method for producing a propylene oligomer of the second embodiment, an oligomer containing a propylene trimer, a propylene tetramer, or a mixture thereof as a main component is isomerized. The term “main component” means that, specifically, the proportion of the propylene trimer, propylene tetramer, or mixture thereof in the oligomer is 50% by mass or more. The proportion of the propylene trimer, propylene tetramer, or mixture thereof contained in the oligomer before being isomerized (material to be isomerized) is preferably 55% by mass or more, more preferably 60% by mass or more, further preferably 65% by mass or more. The oligomer before being isomerized may contain a component other than the propylene trimer and propylene tetramer. Examples of other components include propylene, a propylene dimer, a propylene pentamer and polymers, and modification products produced due to a side reaction, such as cracking, e.g., an olefin having carbon atoms, the number of which is not a multiple of 3. The proportion of the propylene trimer, propylene tetramer, or mixture thereof is preferably 100% by mass, but may be 95% by mass or less, or 90% by mass or less, or 85% by mass or less.

The oligomer before being isomerized, which is a raw material for the isomerization reaction, may be the oligomer obtained as such after the oligomerization of propylene, or may be the fraction obtained by fractional distillation after the oligomerization.

In the present embodiment, the oligomerization may be conducted under the same conditions as those in the oligomerization step in the first embodiment. The reaction temperature which is different from that in the oligomerization step may be lower than 160° C. like the first embodiment, but may be a temperature higher than that in the first embodiment, specifically may be 160° C. or higher and lower than 220° C.

Further, the fractional distillation can be conducted under the same conditions as those in the fractional distillation step in the first embodiment. By conducting the fractional distillation step, the oligomer containing no propylene and light olefin can be isomerized. As a result, the reaction pressure in the present isomerization step can be reduced to lower than the critical pressure of propylene, making it possible to reduce the production cost.

### <Isomerization Step>

The catalyst containing phosphoric acid used in the present step is especially preferably a solid phosphoric acid catalyst from the viewpoint of efficiently obtaining the intended lowly branched propylene oligomer at high selectivity.

Examples of phosphoric acids include orthophosphoric acid, pyrophosphoric acid, and triphosphoric acid, and orthophosphoric acid is preferred. The solid phosphoric acid catalyst contains free phosphoric acid preferably in an amount of 16% by mass or more, and, for improving the catalytic activity, more preferably in a larger amount of free phosphoric acid. Generally, the solid phosphoric acid catalyst contains free phosphoric acid in an amount of 16 to 20% by mass.

Examples of carriers include diatomaceous earth, kaolin, and silica, and diatomaceous earth is preferred.

The carrier may contain an additive for improving the strength of the catalyst. Examples of additives include talc, clay mineral, and iron compounds, such as iron oxide.

The solid phosphoric acid catalyst can be obtained as follows.

It is preferred that phosphoric acid and a carrier are first mixed with each other to obtain a material in a paste form or in a clay form, and the obtained material is formed into a pellet form or a particle form. After the subsequent drying and calcination, the resultant material may be crushed into a particle form.

Then, the material in a paste form or in a clay form is dried, and then calcined to obtain catalyst pellets or catalyst particles.

The temperature for drying is preferably 100 to 300° C., more preferably 150 to 250° C.

The temperature for calcination is preferably 300 to 600° C., more preferably 350 to 500° C.

The catalyst containing phosphoric acid preferably contains water. As examples of methods for causing the catalyst containing phosphoric acid to contain water, there can be mentioned a method in which water vapor is allowed to flow through the above-mentioned catalyst pellets or catalyst particles to cause the catalyst to contain water, and a method in which the catalyst containing phosphoric acid and water are added to the reactor.

The content of phosphoric acid, in terms of phosphoric acid anhydride (P₂O₅), in the solid phosphoric acid catalyst is preferably 30 to 60% by mass, more preferably 40 to 50% by mass.

The content of the carrier in the solid phosphoric acid catalyst is preferably 40 to 80% by mass, more preferably 50 to 60% by mass.

It is preferred that the catalyst containing phosphoric acid is used as a fixed bed catalyst in such a way that a fixed bed reactor is filled with the catalyst.

In the present step, it is preferred that, before initiating the reaction, the water content of the catalyst is controlled. For improving the catalytic activity, it is desired to introduce water.

The reaction pressure in the present isomerization step is less than the critical pressure of propylene. The expression “critical pressure of propylene” means a pressure at the critical point of propylene, specifically 4.66 MPa (absolute pressure). The oligomer containing a propylene trimer, a propylene tetramer, or a mixture thereof as a main component is present in a liquid phase even at a reaction pressure which is less than the critical pressure of propylene. That is, an isomerization reaction in a liquid phase can be conducted even at a pressure which is less than the critical pressure of propylene, and therefore it is possible to improve the reaction efficiency. The reaction pressure in the isomerization step is preferably 3.00 MPa or less, more preferably 2.00 MPa or less, further preferably 1.50 MPa or less, especially preferably 1.00 MPa or less. The reaction pressure is indicated in terms of a gauge pressure. Further, from the viewpoint of the pressure at which the propylene trimer that is a main raw material keeps in a liquid phase, the reaction pressure in the isomerization step is preferably 0.00 MPa or more (atmospheric pressure or more), more preferably 0.05 MPa or more. The reaction pressure is indicated in terms of a gauge pressure.

The present isomerization step is preferably conducted at 160° C. or higher. The reaction temperature in the present step is preferably 160° C. or higher, preferably 160 to 260° C., more preferably 160 to 230° C., further preferably 170 to 220° C., still further preferably 180 to 200° C. By conducting the reaction at 160° C. or higher, the intended propylene oligomer having a low degree of branching can be efficiently obtained in high yield.

The reaction temperature is an average temperature in the reactor, which indicates an average of the temperature of the upstream side and the temperature of the downstream side of the portions of the reactor in contact with the catalyst.

The liquid hourly space velocity in the present isomerization step is preferably 0.1 to 10 h⁻¹, more preferably 0.2 to 8 h⁻¹, further preferably 0.5 to 6 h⁻¹, still further preferably 1 to 4 h⁻¹. When the liquid hourly space velocity is in the above-mentioned range, the intended propylene oligomer having a low degree of branching can be obtained without markedly lowering the yield of the propylene trimer and tetramer.

By conducting the present isomerization step, the propylene oligomer having the intended polymerization degree can be obtained at high selectivity.

The by-product selectivity in the present isomerization step is preferably 20% by mass or less, more preferably 15% by mass or less. By-products are compounds other than the propylene trimer and tetramer which are a product, and the propylene dimer which can be used for producing a product by further conducting the oligomerization step through recycling or the like, specifically, high molecular-weight compounds (propylene pentamer and polymers) produced by a polymerization reaction, modification products produced due to a side reaction, such as cracking, e.g., an olefin having carbon atoms, the number of which is not a multiple of 3, and the like. The by-product selectivity is a content of by-products in the reaction mixture obtained after the isomerization step.

The method for producing a propylene oligomer of the second embodiment may include the fractionation step after the present isomerization step. By subjecting the obtained isomer to fractionation, it is possible to remove impurities and modification products from the isomer.

The distillation conditions in the fractionation step which is conducted after the present isomerization step vary depending on the intended oligomer, but are preferably the conditions described above in connection with <Fractional distillation step> in the first embodiment.

### <Propylene Oligomer Obtained by the Above-Mentioned Production Method>

It is preferred that the propylene oligomer obtained by the production method of the second embodiment has a low degree of branching and has a small Type V olefin content.

When the propylene oligomer obtained by the production method of the second embodiment is a propylene trimer, the propylene trimer preferably has a Type V olefin concentration of 22% by mass or less, more preferably 21% by mass or less, further preferably 20% by mass or less, still further preferably 19% by mass or less, still further preferably 18% by mass or less. The lower limit of the concentration is not limited, but, from the viewpoint of the production efficiency, the lower limit is preferably 10% by mass or more, more preferably 15% by mass or more.

The Type V olefin concentration means a content (% by mass) of the Type V olefin in the propylene trimer, and, with respect to the method for measuring and determining the concentration, the method described in the Examples below is used.

When having a Type V olefin concentration of 23% by mass or less, the propylene trimer can be advantageously used as a raw material for olefin derivatives.

The propylene trimer can contain a Type IV olefin, a Type III olefin, a Type II olefin, and a Type I olefin as well as the Type V olefin.

The Type IV olefin concentration of the propylene trimer in the second embodiment is preferably 50% by mass or more, more preferably 52% by mass or more, further preferably 55% by mass or more. The upper limit of the concentration is not limited, but, from the viewpoint of the production efficiency, the upper limit is preferably 70% by mass or less, more preferably 65% by mass or less.

The Type IV olefin concentration means a content (% by mass) of the Type IV olefin in the propylene trimer, and, with respect to the method for measuring and determining the concentration, the method described in the Examples below is used.

The Type II olefin concentration of the propylene trimer in the second embodiment is preferably 14% by mass or more, preferably 15% by mass or more, more preferably 16% by mass or more, further preferably 18% by mass or more. The upper limit of the concentration is not limited, but, from the viewpoint of the production efficiency, the upper limit is preferably 25% by mass or less, more preferably 22% by mass or less.

The Type II olefin concentration means a content (% by mass) of the Type II olefin in the propylene trimer, and, with respect to the method for measuring and determining the concentration, the method described in the Examples below is used.

The propylene trimer in the second embodiment preferably has a distilling temperature (initial boiling point to end point) of 120 to 160° C., more preferably 125 to 155° C., further preferably 130 to 150° C., still further preferably 130 to 148° C., still further preferably 130 to 145° C., as measured by an atmospheric distillation test method prescribed by JIS K2254:2018. The atmospheric distillation test method is a test method in which a sample is divided into predetermined groups according to the properties, and 100 mL of a sample is subjected to distillation under the conditions to measure an initial boiling point, a distilling temperature, a distillate amount, an end point and the like.

The propylene trimer in the second embodiment preferably has a 50% by volume distilling temperature of 132 to 142° C., more preferably 134 to 140° C., further preferably 135 to 138° C., as measured by an atmospheric distillation test method prescribed by JIS K2254:2018.

When the boiling point (distilling temperature measured by a distillation test) of the propylene trimer is in the above range, the propylene trimer can be advantageously used as a raw material for olefin derivatives as intended.

When the propylene oligomer obtained by the production method of the second embodiment is a propylene tetramer, the propylene tetramer preferably has a Type V olefin concentration of 30% by mass or less, more preferably 26% by mass or less, further preferably 22% by mass or less, still further preferably 20% by mass or less, still further preferably 18% by mass or less. The lower limit of the concentration is not limited, but, from the viewpoint of the production efficiency, the lower limit is preferably 5% by mass or more, more preferably 10% by mass or more.

The Type V olefin concentration means a content (% by mass) of the Type V olefin in the propylene trimer, and, with respect to the method for measuring and determining the concentration, the method described in the Examples below is used.

When having a Type V olefin concentration of 30% by mass or less, the propylene tetramer can be advantageously used as a raw material for olefin derivatives.

The propylene tetramer can contain a Type IV olefin, a Type III olefin, a Type II olefin, and a Type I olefin as well as the Type V olefin.

The Type IV olefin concentration of the propylene tetramer in the second embodiment is preferably 55% by mass or more, more preferably 60% by mass or more, further preferably 63% by mass or more, still further preferably 65% by mass or more. The upper limit of the concentration is not limited, but, from the viewpoint of the production efficiency, the upper limit is preferably 85% by mass or less, more preferably 75% by mass or less.

The Type IV olefin concentration means a content (% by mass) of the Type IV olefin in the propylene tetramer, and, with respect to the method for measuring and determining the concentration, the method described in the Examples below is used.

The propylene tetramer in the second embodiment preferably has a distilling temperature (initial boiling point to end point) of 150 to 230° C., more preferably 155 to 225° C., further preferably 160 to 220° C., still further preferably 165 to 215° C., still further preferably 170 to 210° C., as measured by an atmospheric distillation test method prescribed by JIS K2254:2018.

The propylene tetramer in the second embodiment preferably has a 50% by volume distilling temperature of 175 to 195° C., more preferably 180 to 190° C., further preferably 185 to 190° C., as measured by an atmospheric distillation test method prescribed by JIS K2254:2018.

When the boiling point (distilling temperature measured by a distillation test) of the propylene tetramer is in the above range, the propylene tetramer can be advantageously used as a raw material for olefin derivatives as intended.

### Third Embodiment

The third embodiment of the present disclosure is a propylene oligomer containing a propylene tetramer having a 4,6,6-trimethyl-3-nonene concentration of 30% by mass or more. Further, the third embodiment of the present disclosure is a technique that relates to a method for producing the propylene oligomer, which includes the step of oligomerizing propylene in the presence of a catalyst containing crystalline molecular sieve, wherein when the BET specific surface area of the crystalline molecular sieve, as obtained by a nitrogen adsorption method, is taken as “a” [m²/g] and the micropore specific surface area of the crystalline molecular sieve, as obtained by subjecting an adsorption isotherm measured by a nitrogen adsorption method to analysis in accordance with a t-plot method, is taken as “b” [m²/g], a/b is 1.8 or less.

In the present invention, the term “micropores” means pores having a diameter of 2 nm or less among the pores of the crystalline molecular sieve. The term “pores” is a collective term which refers to all of micropores, mesopores, and macropores defined by IUPAC, and specifically indicates pores measured by nitrogen adsorption. The term “BET specific surface area” means a specific surface area of the crystalline molecular sieve, as determined by a BET analysis using an adsorption isotherm measured by a nitrogen adsorption method. The term “micropore specific surface area” means a specific surface area obtained by subjecting an adsorption isotherm measured by a nitrogen adsorption method to analysis in accordance with a t-plot method. The micropore specific surface area of the crystalline molecular sieve may be a value determined directly by an analysis in accordance with a t-plot method, and may be a value determined by obtaining a specific surface area of pores other than micropores by an analysis in accordance with a t-plot method, and subtracting the specific surface area of pores other than micropores from the above-mentioned BET specific surface area.

Hereinbelow, the third embodiment is described in detail.

### [Propylene Oligomer]

The propylene oligomer of the third embodiment contains a propylene tetramer having a 4,6,6-trimethyl-3-nonene concentration of 30% by mass or more.

In the present disclosure, 4,6,6-trimethyl-3-nonene includes geometrical isomers represented by the chemical formulae (I) and (II) below. 4,6,6-Trimethyl-3-nonene corresponds to Type IV olefin shown in Table 1 above.

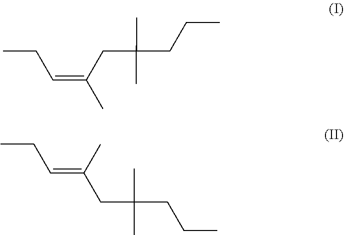

A highly branched isomer is highly active in a reaction, for example, a Koch reaction or an alkylation reaction. The reactivity of an isomer is considered to vary depending on the steric environment surrounding the double bond of the isomer. Further, a product produced using an oligomer containing a large amount of a highly branched isomer has a viscosity lower than the viscosity of a product produced using an oligomer containing a large amount of a linear or lowly branched isomer. Such an oligomer not only has an advantageous phenomenon about the viscosity but also can be expected to be improved in detergency, biodegradability and the like in the use as a surfactant.

That is, the propylene oligomer of the present disclosure contains 4,6,6-trimethyl-3-nonene which is a highly branched propylene oligomer at a high concentration, and therefore is useful as a raw material for a surfactant and the like.

In the propylene oligomer of the third embodiment, the concentration of 4,6,6-trimethyl-3-nonene in the propylene tetramer is 30% by mass or more, preferably 35% by mass or more, more preferably 40% by mass or more. The upper limit of the concentration is not particularly limited, and is especially preferably 100% by mass, but may be 90% by mass or less, 80% by mass or less, or 70% by mass or less.

With respect to the method for measuring and determining the 4,6,6-trimethyl-3-nonene concentration, the method described in the Examples below is used.

In the third embodiment, the propylene tetramer can contain a Type IV olefin other than 4,6,6-trimethyl-3-nonene, a Type V olefin, a Type III olefin, a Type II olefin, and a Type I olefin. In the present embodiment, with respect to the respective contents of the Type IV olefin, Type V olefin other than the 4,6,6-trimethyl-3-nonene, Type III olefin, Type II olefin, and Type I olefin, there is no particular limitation.

The propylene tetramer in the third embodiment preferably has a distilling temperature (initial boiling point to end point) of 150 to 230° C., more preferably 155 to 225° C., further preferably 160 to 220° C., still further preferably 165 to 215° C., still further preferably 170 to 210° C., as measured by an atmospheric distillation test method prescribed by JIS K2254:2018. The atmospheric distillation test method is a test method in which a sample is divided into predetermined groups according to the properties, and 100 mL of a sample is subjected to distillation under the conditions to measure an initial boiling point, a distilling temperature, a distillate amount, an end point and the like.

The propylene tetramer in the third embodiment preferably has a 50% by volume distilling temperature of 175 to 195° C., more preferably 180 to 190° C., further preferably 185 to 190° C., as measured by an atmospheric distillation test method prescribed by JIS K2254.2018.

When the boiling point (distilling temperature measured by a distillation test) of the propylene tetramer is in the above range, the propylene tetramer can be advantageously used as a raw material for olefin derivatives as intended.

The propylene oligomer of the third embodiment can contain a propylene oligomer other than the propylene tetramer. Examples of propylene oligomers other than the propylene tetramer include a propylene dimer, trimer, pentamer and polymers. Further, the propylene oligomer of the third embodiment can contain modification products produced due to a side reaction, such as cracking, e.g., an olefin having carbon atoms, the number of which is not a multiple of 3, and the like.

It is preferred that the propylene oligomer of the third embodiment contains a propylene tetramer in an amount of 3% by mass or more. When the content of the propylene tetramer in the propylene oligomer is 3% by mass or more, consequently, the propylene oligomer can contain 4,6,6-trimethyl-3-nonene at a high concentration. The content of the propylene tetramer is more preferably 5% by mass or more, further preferably 10% by mass or more, especially preferably 15% by mass or more. Further, the upper limit of the content of the propylene tetramer is not particularly limited, but may be 80% by mass or less, 70% by mass or less, or 60% by mass or less.

When the below-mentioned fractional distillation step is not performed, the content of the propylene dimer in the propylene oligomer is preferably 20% by mass or more, more preferably 30% by mass or more.

Further, when the below-mentioned fractional distillation step is not performed, the content of the propylene trimer in the propylene oligomer is preferably 15% by mass or more, more preferably 30% by mass or more. On the other hand, from the viewpoint of increasing the content of the propylene tetramer, the content of the propylene trimer in the propylene oligomer is preferably 60% by mass or less, more preferably 40% by mass or less.

### [Method for Producing a Propylene Oligomer]

### <Oligomerization Step>

The method for producing a propylene oligomer of the third embodiment includes the step of oligomerizing propylene in the presence of a catalyst containing crystalline molecular sieve, wherein when the BET specific surface area of the crystalline molecular sieve, as obtained by a nitrogen adsorption method, is taken as “a” [m²/g] and the micropore specific surface area of the crystalline molecular sieve, as obtained by subjecting an adsorption isotherm measured by a nitrogen adsorption method to analysis in accordance with a t-plot method, is taken as “b” [m²/g], a/b is 1.8 or less.

By the above-mentioned oligomerization step, a propylene oligomer containing a propylene tetramer having a 4,6,6-trimethyl-3-nonene concentration of 30% by mass or more can be produced That is, by conducting oligomerization using crystalline molecular sieve in which a/b is 1.8 or less as a catalyst, an oligomer having a specific structure can be obtained at high selectivity.

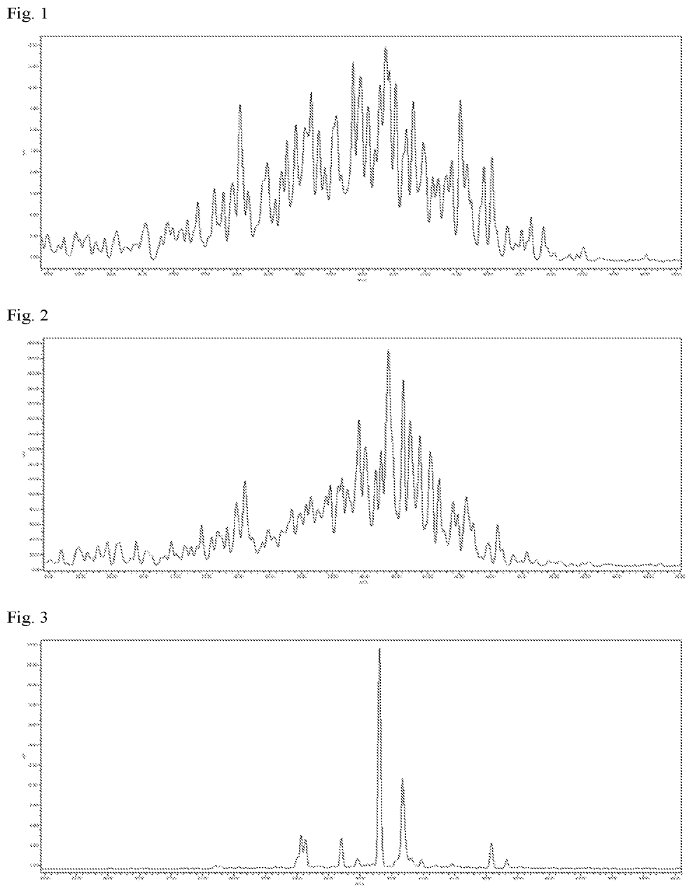

FIGS. 1 to 3 are GC charts of propylene oligomers having 12 carbon atoms obtained by oligomerization conducted in the presence of different catalysts. When using a solid phosphoric acid catalyst (FIG. 1, the below-mentioned Comparative Example 10) or crystalline molecular sieve in which the ratio of the BET specific surface area to the micropore specific surface area (a/b) is more than 1.8 (FIG. 2, the below-mentioned Comparative Example 7) as a catalyst, a number of peaks can be found. That is, the produced propylene tetramer contains a number of types of isomers. On the other hand, when using crystalline molecular sieve in which the ratio of the BET specific surface area to the micropore specific surface area (a/b) is 1.8 or less (FIG. 3, the below-mentioned Example 5) as a catalyst, the number of peaks is extremely small and specific strong peaks are detected. A further analysis showed that the two strongest peaks (40.3 minutes and 40.7 minutes) in FIG. 3 are ascribed to 4,6,6-trimethyl-3-nonene. As apparent from the above, by using crystalline molecular sieve having a large micropore specific surface area, a propylene oligomer containing a propylene tetramer having a specific structure (4,6,6-trimethyl-3-nonene) at a high concentration can be produced.

The reason that 4,6,6-trimethyl-3-nonene is produced at high selectivity is not clear, but is presumed as follows.

In the oligomerization using a solid phosphoric acid catalyst or a solid acid catalyst having a large average pore diameter, such as silica alumina, a reaction proceeds without steric control. For this reason, a main route of the reaction is such a route that propylene is added to a propylene trimer having various isomers to produce a propylene tetramer. As a result of the reaction, a propylene tetramer having isomers of a wider variety of types than the propylene trimer is produced. Meanwhile, in the case of crystalline molecular sieve in which the ratio of the BET specific surface area to the micropore specific surface area (a/b) is more than 1.8, that is, the proportion of the micropore specific surface area is small, the crystalline properties of the molecular sieve are poor, and the proportion of micropores is small, and therefore an oligomerization reaction is likely to proceed in a place other than the pores derived from the crystal structure. Accordingly, steric control due to the micropores is unlikely to be caused, and a main reaction route is an oligomerization reaction such that propylene is added to a propylene trimer having various isomers. For this reason, like the above-mentioned oligomerization using a solid acid catalyst, a propylene tetramer having various isomers is produced. On the other hand, in the case of crystalline molecular sieve in which the ratio of the BET specific surface area to the micropore specific surface area (a/b) is 1.8 or less, the proportion of micropores is large, and hence it is presumed that form selectivity due to the micropores is exhibited, so that an oligomerization reaction is likely to be caused inside the micropores. It is considered that the form selectivity causes the following reaction route to selectively proceed: reaction route in which propylene dimers which are easily produced, i.e., 2-methyl-1-pentene and 2-methyl-2-pentene are first produced, and then these propylene dimers undergo dimerization to produce a propylene tetramer, i.e., 4,6,6-trimethyl-3-nonene.

From the viewpoint of obtaining a propylene oligomer having a specific structure at high selectivity, with respect to the crystalline molecular sieve contained in the catalyst used in the present step, the ratio of the BET specific surface area (a) to the micropore specific surface area (b), i.e., a/b is preferably 1.75 or less, more preferably 1.7 or less, further preferably 1.65 or less.

The BET specific surface area measured by a nitrogen adsorption method conducted in the present step is a value obtained by making an analysis at a relative pressure in the range of 0.005 to 0.1. Such an analysis is made in order to properly evaluate the specific surface area of the crystalline molecular sieve having micropores based on the BET theory.

Further, the micropore specific surface area measured by a t-plot method conducted in the present step is a value obtained by making an analysis under conditions that the average thickness (t) of the nitrogen which has adsorbed is in the range of 5 to 6.5 Å. Such an analysis is made in order to reduce the influence of the mesopores derived from a binder and the like so as to properly evaluate the micropore specific surface area derived from the crystalline molecular sieve based on the t-plot theory.

As the crystalline molecular sieve, zeolite is preferred. As the crystalline molecular sieve, 10-membered ring zeolite is especially preferred.

Examples of the 10-membered ring zeolite include those of an MFI type (another name: ZSM-5), an MFS type (another name: ZSM-57), a TON type (another name: ZSM-22), an MTT type (another name: ZSM-23), an MEL type (another name: ZSM-11), an FER type, an MRE type (another name: ZSM-48), and an MWW type (another name: MCM-22). Of these, MFI-type zeolite is more preferred.

With respect to the crystalline molecular sieve, the ratio of the pore volume and the micropore volume (pore volume/micropore volume) is preferably 2.0 to 5.5. When the ratio of the micropore volume to the pore volume is in the above range, the proportion of micropores is large, and it is likely that form selectivity is exhibited. For this reason, a reaction of a specific route is likely to selectively proceed, increasing the 4,6,6-trimethyl-3-nonene concentration of the tetramer. The ratio of the micropore volume to the pore volume is more preferably 3.0 to 5.0, further preferably 3.5 to 4.5.

From the viewpoint of allowing the reaction to more efficiently proceed, the crystal diameter of the 10-membered ring zeolite as observed by a SEM (scanning electron microscope) is preferably 1 μm or less, more preferably 0.5 μm or less, further preferably 0.1 μm or less.

From the viewpoint of allowing the reaction to efficiently proceed, the silicon/aluminum molar ratio (Si/Al) of the 10-membered ring zeolite is preferably 100 or less, more preferably 50 or less, further preferably 25 or less.

From the viewpoint of allowing the reaction to efficiently proceed, the acid amount of the 10-membered ring zeolite as measured by NH₃-TPD is preferably 150 μmol/g or more, more preferably 200 μmol/g or more, further preferably 250 μmol/g or more.

For improving the moldability for catalyst, a binder may be used when molding zeolite. As the binder, a metal oxide, such as alumina, silica, or clay mineral, can be used, and, from the viewpoint of the effect on the mechanical strength, cost, and acid site and the like, the binder is preferably alumina. As the amount of the binder used is reduced, the amount of the zeolite which is an active site is increased, and therefore the amount of the binder is preferably 50% by mass or less, more preferably 30% by mass or less, further preferably 20% by mass or less.

It is preferred that the catalyst containing crystalline molecular sieve is used as a fixed bed catalyst in such a way that a fixed bed reactor is filled with the catalyst.

In the oligomerization step, it is preferred that, before initiating the reaction, a pretreatment for removing impurities from the catalyst is conducted. As a method for pretreatment, preferred is a method in which a gas that is inert with respect to the present oligomerization reaction, such as nitrogen or LPG, is increased in temperature and a flow of the gas at a high temperature is passed through the reactor.

The temperature for pretreatment is preferably 100 to 500° C., more preferably 150 to 400° C., further preferably 150 to 300° C. The time for pretreatment varies depending on the size of the reactor, but is preferably 1 to 20 hours, more preferably 2 to 10 hours.

Further, it is preferred that, before initiating the reaction, the water content of the catalyst is controlled. In the case of the catalyst containing crystalline molecular sieve, for improving the catalytic activity, it is preferred to remove water, and, for increasing the life of the catalyst, it is preferred to add water. As a method for removing water, the above-mentioned pretreatment method is preferably used.

Then, propylene is introduced.

The propylene introduced may be used in the form of a mixture with a gas which is inert with respect to the present oligomerization reaction. The concentration of propylene in the reaction mixture except the catalyst is preferably 55% by volume or more, more preferably 60% by volume or more, further preferably 65% by volume or more, still further preferably 70% by volume or more.

The reaction temperature in the oligomerization step in the present embodiment is preferably lower than 220° C., more preferably 90° C. or higher and lower than 210° C., further preferably 120° C. or higher and lower than 200° C., especially preferably 125° C. or higher and 180° C. or lower. By conducting the reaction at lower than 220° C., the above-mentioned propylene oligomer can be obtained in high yield while suppressing degradation of the catalyst.

The reaction temperature is an average temperature in the reactor, which indicates an average of the temperature of the upstream side and the temperature of the downstream side of the portions of the reactor in contact with the catalyst.

The liquid hourly space velocity in the oligomerization step is preferably 5 h⁻¹ or less, more preferably 4 h⁻¹ or less, further preferably 3 h⁻¹ or less, still further preferably 2 h⁻¹ or less. When the liquid hourly space velocity is 5 h⁻¹ or less, the above-mentioned propylene oligomer can be obtained in high yield.

The preliminary reaction time in the oligomerization step is preferably 100 hours or more, more preferably 200 hours or more, further preferably 250 hours or more, still further preferably 270 hours or more. By providing the preliminary reaction time before obtaining the reaction product, the catalyst can be stabilized, so that the above-mentioned propylene oligomer can be obtained in high yield.

The propylene conversion in the present step is preferably 50 to 99.9%, more preferably 50 to 99%, further preferably 60 to 97%, still further preferably 70 to 95%.

In the present step, for the purpose of removing the heat from the reactor or reducing the amount of the unreacted propylene, recycling can be made by permitting the unreacted propylene and light oligomers produced by the reaction, which are discharged from the outlet of the reactor, to go back to the reactor. As mentioned above, in the present embodiment, light oligomers include mainly a dimer of propylene (2-methyl-1-pentene and 2-methyl-2-pentene or the like). Thus, recycling can increase not only the amount of the propylene tetramer produced but also the amount of the 4,6,6-trimethyl-3-nonene produced. When recycling is conducted, from the viewpoint of the production efficiency, the ratio of the fresh feed (propylene as a raw material) and the recycle (the unreacted propylene and light oligomers) (R/F) is preferably 0.1 to 10, more preferably 0.3 to 6, further preferably 1 to 3.

### <Fractional Distillation Step>

The method for producing the propylene oligomer of the third embodiment may further include the fractional distillation step of obtaining a fraction containing a propylene tetramer. The present fractional distillation step is conducted for removing low molecular-weight compounds (a propylene dimer, a propylene trimer) and high molecular-weight compounds (a pentamer and polymers), which are by-products produced by oligomerization, modification products produced due to a side reaction, such as cracking, e.g., an olefin having carbon atoms, the number of which is not a multiple of 3, and the like.

Conditions for the fractional distillation vary depending on the pressure, the size of the distillation apparatus, the number of plates of the distillation column, and the like, and further vary depending on the production efficiency, the intended purity, and the use, but it is preferred that the fractional distillation is conducted under conditions such that an olefin having 12 carbon atoms which is a propylene tetramer is obtained.

When an olefin having 12 carbon atoms which is a propylene tetramer is mainly obtained, the distilling set temperature for the distillation under atmospheric pressure (1 atm) is preferably 150 to 230° C., more preferably 160 to 220° C., further preferably 170 to 210° C., still further preferably 190 to 210° C.

In the third embodiment, from the viewpoint of obtaining a propylene tetramer having a specific structure at a high concentration, it is preferred that the isomerization step described above in connection with the first embodiment is not performed.

In the third embodiment, after conducting the oligomerization step, or after conducting the fractional distillation step, the fractionation step can be performed. By performing fractionation, it is possible to remove impurities and modification products.

The distillation conditions in the fractionation step are preferably the conditions described above in connection with the fractional distillation step.

### EXAMPLES

Hereinbelow, the present disclosure will be described in more detail with reference to the following Examples, which should not be construed as limiting the technique of the present disclosure.

In the following Examples and Comparative Examples, the reaction pressure and the pressure during the reaction are indicated in terms of a gauge pressure.

### Examples 1 to 3 and Comparative Examples 1 to 5

The analysis methods for the propylene oligomers obtained in the Examples and Comparative Examples are as follows.

### (1) Composition (Proportion of Each Olefin Type)

With respect to the propylene trimers in the Examples and Comparative Examples, using a nuclear magnetic resonance apparatus (NMR) ECA500 (manufactured by JEOL LTD.), a proportion of each olefin type was determined as described below.

The propylene trimers obtained in the Examples and Comparative Examples were individually dissolved in deuterochloroform (chloroform-d), and subjected to ¹H-NMR measurement. In the NMR spectrum obtained using chloroform (7.26 ppm) as a reference, a peak appearing at 5.60 to 5.90 ppm was ascribed to Type I (vinyl type) olefin, a peak appearing at 4.58 to 4.77 ppm was ascribed to Type III (vinylidene type) olefin, a peak appearing at 5.30 to 5.60 ppm was ascribed to Type II olefin, and a peak appearing at 4.77 to 5.30 ppm was ascribed to Type IV olefin, and, from the area ratio between these peaks, the ratio between the olefin types was calculated. Further, from the area ratio of the above peaks and the other peaks, a total amount of Type I (vinyl type) olefin, Type III (vinylidene type) olefin, Type II olefin, and Type IV olefin was calculated, and a content of the remaining Type V olefin was calculated. A proportion of each olefin type was calculated by multiplying the total amount of Type I (vinyl type) olefin, Type III (vinylidene type) olefin, Type II olefin, and Type IV olefin by the above-mentioned ratio between the olefin types. The peaks were ascribed to the above-mentioned olefin types according to Stehling et al., Anal. Chem., 38 (11), pp. 1467 to 1479 (1966).

(2) Composition (selectivity; proportion of each of the oligomers having polymerization degrees)

The selectivity of the propylene oligomer in each step in the Examples and Comparative Examples (proportion of each of the oligomers having polymerization degrees) was determined using a gas chromatography apparatus (6850 Network GC System, manufactured by Aglent Technologies, Inc.) as described below. As a column, DB-PETRO, manufactured by Aglent Technologies, Inc., (100 m×0.250 mm×0.50 μm) was used. As a carrier gas, helium was used, and the flow rate was 2.5 mL/minute. The injection temperature was 250° C., and the split ratio was 100. The reaction mixture was charged in the state of maintaining the oven temperature at 50° C., and maintained at 50° C. for 10 minutes. Then, the temperature of the oven was increased to 300° C. at a temperature increase rate of 3.13° C./minute, and the individual components were identified. A peak appearing at 5.6 to 6.2 minutes was ascribed to propylene, a peak appearing at 8.0 to 11.8 minutes was ascribed to a propylene dimer, a peak appearing at 21.9 to 29.2 minutes was ascribed to a propylene trimer, a peak appearing at 36.7 to 43.9 minutes was ascribed to a propylene tetramer, and the other peaks were ascribed to by-products.

### Production Example 1 (Preparation of a Solid Phosphoric Acid Catalyst)

34 Parts by mass of diatomaceous earth (Silica Queen S, manufactured by Chuo Silika Co., Ltd.) as a carrier and 66 parts by mass of orthophosphoric acid (manufactured by FUJIFILM Wako Pure Chemical Corporation; special grade reagent; purity: 85% or more) were weighed, and charged into a kneader and well kneaded with each other. The resultant mixture in a clay form was placed in an extrusion molding machine, and extruded into pellets in the shape of a 4.5 mmø cylinder.

The obtained pellets were placed in a muffle furnace, and increased in temperature from room temperature at a rate of 10° C./min, and dried at 200° C. for 3 hours, and then further increased in temperature at a rate of 10° C./min, and calcined at 400° C. for 2 hours. All these operations were performed in an air flow. Then, the flowing gas was changed to air containing water vapor in an amount of about 20%, and further maintained at a temperature of 400° C. for one hour. After these operations, the temperature was reduced to room temperature, obtaining a solid phosphoric acid catalyst in a pellet form.

The obtained solid phosphoric acid catalyst in a pellet form was crushed and sieved using sieves of a 6 mesh size and a 9 mesh size, obtaining a solid phosphoric acid catalyst in a particle form such that the particles are uniform.

### Example 1 (Production of Propylene Oligomer (1))

### (1) Oligomerization Step

40 cc of a zeolite catalyst (MFI type (another name: ZSM-5); 10-membered ring; HSZ-822HOD1A, manufactured by Tosoh Corp.; catalyst diameter: 1.5 mmø; catalyst length: 3 mm; extrusion molded article in a cylindrical shape) and 40 cc of alumina balls (2 mmø; spherical; SSA-995, manufactured by Nikkato Corporation) were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, propylene was introduced so that the reaction pressure became 6.5 MPa and the feed rate became 60 cc/hour (LHSV=1.5 h⁻¹). For stabilizing the catalyst, a reaction was conducted for 37 days (888 hours), and then the reaction mixture was withdrawn. The average reaction temperature of the reaction tube was 151.9° C. Further, the propylene conversion was 93.7%.

### (2) Fractional Distillation Step

The reaction mixture obtained in the oligomerization step was subjected to fractional distillation to obtain a fraction containing mainly a propylene trimer. The distillation set temperature was 130 to 145° C.

### (3) Isomerization Step

A fixed bed reaction tube made of stainless steel was filled with 20 cc of the solid phosphoric acid catalyst obtained in Production Example 1.

Then, the fraction obtained in the fractional distillation step was introduced so that the feed rate became 30 cc/hour (LHSV=1.5 h⁻¹). For preventing the solid phosphoric acid catalyst from lowering in the activity, water was simultaneously introduced in an amount of 100 ppm by mass, based on the mass of the raw material. A reaction was conducted for 72 days (1,733 hours), and then an isomerization reaction mixture was obtained. The obtained isomerization reaction mixture was subjected to fractionation at a distillation set temperature of 130 to 145° C. to obtain a propylene oligomer (1). The average reaction temperature was 193.3° C., and the pressure during the reaction was 0.9 MPa. The results of the analysis made with respect to the obtained propylene oligomer (1) are shown in Table 2.

### Example 2 (Production of Propylene Oligomer (2))

### (1) Oligomerization Step

A fixed bed reaction tube made of stainless steel was filled with 60 cc of the solid phosphoric acid catalyst obtained in Production Example 1.

Then, propylene was introduced so that the reaction pressure became 6.5 MPa and the feed rate became 90 cc/hour (LHSV=1.5 h⁻¹). For preventing the solid phosphoric acid catalyst from lowering in the activity, water was simultaneously introduced in an amount of 25 ppm by mass, based on the mass of the raw material. A reaction was conducted for 38 days (912 hours), and then the reaction mixture was withdrawn. The average reaction temperature was 145.1° C. Further, the propylene conversion was 94.0%.

### (2) Fractional Distillation Step

The reaction mixture obtained in the oligomerization step was subjected to fractional distillation to obtain a fraction containing mainly a propylene trimer. The distillation set temperature was 130 to 145° C.

### (3) Isomerization Step

A fixed bed reaction tube made of stainless steel was filled with 20 cc of the solid phosphoric acid catalyst obtained in Production Example 1.

Then, the fraction obtained in the fractional distillation step was introduced so that the feed rate became 30 cc/hour (LHSV=1.5 h⁻¹). For preventing the solid phosphoric acid catalyst from lowering in the activity, water was simultaneously introduced in an amount of 70 ppm by mass, based on the mass of the raw material. A reaction was conducted for 77 days (1,841 hours), and then an isomerization reaction mixture was obtained. The obtained isomerization reaction mixture was subjected to fractionation at a distillation set temperature of 130 to 145° C. to obtain a propylene oligomer (2). The average reaction temperature was 184.5° C., and the pressure during the reaction was 0.8 MPa. The results of the analysis made with respect to the obtained propylene oligomer (2) are shown in Table 2.

### Example 3 (Production of Propylene Oligomer (3))

### (1) Oligomerization Step

A fixed bed reaction tube made of stainless steel was filled with 60 cc of the solid phosphoric acid catalyst obtained in Production Example 1.

Then, propylene was introduced so that the reaction pressure became 6.5 MPa and the feed rate became 90 cc/hour (LHSV=1.5 h⁻¹). For preventing the solid phosphoric acid catalyst from lowering in the activity, water was simultaneously introduced in an amount of 175 ppm by mass, based on the mass of the raw material. A reaction was conducted for 6 days (132 hours), and then the reaction mixture was withdrawn. The average reaction temperature was 160.6° C. Further, the propylene conversion was 95.4%.

### (2) Fractional Distillation Step

The reaction mixture obtained in the oligomerization step was subjected to fractional distillation to obtain a fraction containing mainly a propylene trimer. The distillation set temperature was 130 to 145° C.

### (3) Isomerization Step

A fixed bed reaction tube made of stainless steel was filled with 20 cc of the solid phosphoric acid catalyst obtained in Production Example 1.

Then, the fraction obtained in the fractional distillation step was introduced so that the feed rate became 30 cc/hour (LHSV=1.5 h⁻¹). For preventing the solid phosphoric acid catalyst from lowering in the activity, water was simultaneously introduced in an amount of 391 ppm by mass, based on the mass of the raw material. A reaction was conducted for 23 days (546 hours), and then an isomerization reaction mixture was obtained. The obtained isomerization reaction mixture was subjected to fractionation at a distillation set temperature of 130 to 145° C. to obtain a propylene oligomer (3). The average reaction temperature was 183.8° C., and the pressure during the reaction was 0.8 MPa. The results of the analysis made with respect to the obtained propylene oligomer (3) are shown in Table 2.

### Comparative Example 1 (Production of Propylene Oligomer (4))

### (1) Oligomerization Step

40 cc of a zeolite catalyst (MFI type (another name: ZSM-5); 10-membered ring; HSZ-822HOD1A, manufactured by Tosoh Corp.; catalyst diameter: 1.5 mmø; catalyst length: 3 mm; extrusion molded article in a cylindrical shape) and 40 cc of alumina balls (2 mmø; spherical; SSA-995, manufactured by Nikkato Corporation) were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, propylene was introduced so that the reaction pressure became 6.5 MPa and the feed rate became 60 cc/hour (LHSV=1.5 h⁻¹). For stabilizing the catalyst, a reaction was conducted for 37 days (888 hours), and then the reaction mixture was withdrawn. The obtained oligomerization reaction mixture was subjected to fractionation at a distillation set temperature of 130 to 145° C. to obtain a propylene oligomer (4). The average reaction temperature of the reaction tube was 151.9° C. Further, the propylene conversion was 93.7%. The results of the analysis made with respect to the propylene oligomer (4) are shown in Table 2.

### Comparative Example 2 (Production of Propylene Oligomer (5))

### (1) Oligomerization Step

A fixed bed reaction tube made of stainless steel was filled with 60 cc of the solid phosphoric acid catalyst obtained in Production Example 1.

Then, propylene was introduced so that the reaction pressure became 6.5 MPa and the feed rate became 90 cc/hour (LHSV=1.5 h⁻¹). For preventing the solid phosphoric acid catalyst from lowering in the activity, water was simultaneously introduced in an amount of 25 ppm by mass, based on the mass of the raw material. A reaction was conducted for 38 days (912 hours), and then the reaction mixture was withdrawn. The obtained oligomerization reaction mixture was subjected to fractionation at a distillation set temperature of 130 to 145° C. to obtain a propylene oligomer (5). The average reaction temperature was 145.1° C. Further, the propylene conversion was 94.0%. The results of the analysis made with respect to the propylene oligomer (5) are shown in Table 2.

### Comparative Example 3 (Production of Propylene Oligomer (6))

### (1) Oligomerization Step

A fixed bed reaction tube made of stainless steel was filled with 60 cc of the solid phosphoric acid catalyst obtained in Production Example 1.

Then, propylene was introduced so that the reaction pressure became 6.5 MPa and the feed rate became 90 cc/hour (LHSV=1.5 h⁻¹). For preventing the solid phosphoric acid catalyst from lowering in the activity, water was simultaneously introduced in an amount of 25 ppm by mass, based on the mass of the raw material. A reaction was conducted for 38 days (912 hours), and then the reaction mixture was withdrawn. The average reaction temperature was 145.1° C. Further, the propylene conversion was 94.0%.

### (2) Fractional Distillation Step

The reaction mixture obtained in the oligomerization step was subjected to fractional distillation to obtain a fraction containing mainly a propylene trimer. The distillation set temperature was 130 to 145° C.

### (3) Isomerization Step

40 cc of a zeolite catalyst (MFI type (another name: ZSM-5); 10-membered ring; HSZ-822HOD1A, manufactured by Tosoh Corp.; catalyst diameter: 1.5 mmø; catalyst length: 3 mm; extrusion molded article in a cylindrical shape) and 40 cc of alumina balls (2 mmø; spherical; SSA-995, manufactured by Nikkato Corporation) were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, the fraction obtained in the fractional distillation step was introduced so that the feed rate became 60 cc/hour (LHSV=1.5 h⁻¹). A reaction was conducted for 6.5 days (156 hours), and then an isomerization reaction mixture was obtained. The obtained isomerization reaction mixture was subjected to fractionation at a distillation set temperature of 130 to 145° C. to obtain a propylene oligomer (6). The average reaction temperature was 190.1° C., and the pressure during the reaction was 0.9 MPa. The results of the analysis made with respect to the obtained propylene oligomer (6) are shown in Table 2.

### Comparative Example 4 (Production of Propylene Oligomer (7))

### (1) Oligomerization Step

A fixed bed reaction tube made of stainless steel was filled with 60 cc of the solid phosphoric acid catalyst obtained in Production Example 1.

Then, propylene was introduced so that the reaction pressure became 6.5 MPa and the feed rate became 90 cc/hour (LHSV=1.5 h⁻¹). For preventing the solid phosphoric acid catalyst from lowering in the activity, water was simultaneously introduced in an amount of 100 ppm by mass, based on the mass of the raw material. A reaction was conducted for 4.5 days (108 hours), and then the reaction mixture was withdrawn. The obtained oligomerization reaction mixture was subjected to fractionation at a distillation set temperature of 130 to 145° C. to obtain a propylene oligomer (7). The average reaction temperature was 198.1° C., and the propylene conversion was 99.3%. The results of the analysis made with respect to the obtained propylene oligomer (7) are shown in Table 2.

### Comparative Example 5 (Production of Propylene Oligomer (8))

### (1) Oligomerization Step

A fixed bed reaction tube made of stainless steel was filled with 60 cc of the solid phosphoric acid catalyst obtained in Production Example 1.

Then, propylene was introduced so that the reaction pressure became 6.5 MPa and the feed rate became 90 cc/hour (LHSV=1.5 h⁻¹). For preventing the solid phosphoric acid catalyst from lowering in the activity, water was simultaneously introduced in an amount of 175 ppm by mass, based on the mass of the raw material. A reaction was conducted for 6 days (132 hours), and then the reaction mixture was withdrawn. The obtained oligomerization reaction mixture was subjected to fractionation at a distillation set temperature of 130 to 145° C. to obtain a propylene oligomer (8). The average reaction temperature was 160.6° C., and the propylene conversion was 95.4%. The results of the analysis made with respect to the obtained propylene oligomer (8) are shown in Table 2.

TABLE 2

| | | | | | | Compar- | Compar- | Compar- | Compar- | Compar- |
|---|---|---|---|---|---|---|---|---|---|---|
| | | | | | | ative | ative | ative | ative | ative |
| | | | Example 1 | Example 2 | Example 3 | Example 1 | Example 2 | Example 3 | Example 4 | Example 5 |
| Oligo- | Catalyst | | Zeolite | Solid | Solid | Zeolite | Solid | Solid | Solid | Solid |
| merization | | | (MFI-type) | phosphoric | phosphoric | (MFI-type) | phosphoric | phosphoric | phosphoric | phosphoric |
| step | | | | acid | acid | | acid | acid | acid | acid |
| | Reaction pressure | | 6.5 MPa | 6.5 MPa | 6.5 MPa | 6.5 MPa | 6.5 MPa | 6.5 MPa | 6.5 MPa | 6.5 MPa |
| | Reaction temperature | | 151.9° C. | 145.1° C. | 160.6° C. | 151.9° C. | 145.1° C. | 145.1° C. | 198.1° C. | 160.6° C. |
| | Space velocity | | 1.5 hr⁻¹ | 1.5 hr⁻¹ | 1.5 hr⁻¹ | 1.5 hr⁻¹ | 1.5 hr⁻¹ | 1.5 hr⁻¹ | 1.5 hr⁻¹ | 1.5 hr⁻¹ |
| | Reaction time | | 888 hr | 912 hr | 132 hr | 888 hr | 912 hr | 912 hr | 108 hr | 132 hr |
| | (including | | | | | | | | | |
| | preliminary | | | | | | | | | |
| | reaction time) | | | | | | | | | |
| | Propylene conversion | | 93.7% | 94.0% | 95.4% | 93.7% | 94.0% | 94.0% | 99.3% | 95.4% |
| | By-product | | 3.2% | 7.1% | 8.2% | 3.2% | 7.1% | 7.1% | 20.9% | 8.2% |
| | selectivity (mass %) | | | | | | | | | |
| Fractional | Distillation | | 130-145° C. | 130-145° C. | 130-145° C. | 130-145° C. | 130-145° C. | 130-145° C. | 130-145° C. | 130-145° C. |
| distillation | temperature | | | | | | | | | |
| step | Compo- | Trimer | 97.5% | 97.5% | 97.0% | 97.5% | 97.5% | 97.5% | 97.5% | 97.0% |
| | sition | Tetramer | 0.0% | 0.0% | 0.0% | 0.0% | 0.0% | 0.0% | 0.0% | 0.0% |
| | (mass %) | By- | 2.5% | 2.5% | 3.0% | 2.5% | 2.5% | 2.5% | 2.5% | 3.0% |
| | | products | | | | | | | | |
| Isom- | Catalyst | | Solid | Solid | Solid | (No | (No | Zeolite | (No | (No |
| erization | | | phosphoric | phosphoric | phosphoric | isom- | isom- | (MFI-type) | isom- | isom- |
| step | | | acid | acid | acid | erization | erization | | erization | erization |
| | Reaction pressure | | 0.9 MPa | 0.8 MPa | 0.8 MPa | step) | step) | 0.9 MPa | step) | step) |
| | Reaction temperature | | 193.3° C. | 184.5° C. | 183.8° C. | | | 190.1° C. | | |
| | Space velocity | | 1.5 hr⁻¹ | 1.5 hr⁻¹ | 1.5 hr⁻¹ | | | 1.5 hr⁻¹ | | |
| | Reaction time | | 1733 hr | 1841 hr | 546 hr | | | 156 hr | | |
| | Compo- | Dimer | 1.0% | 0.5% | 0.2% | | | 0.3% | | |
| | sition | Trimer | 89.3% | 91.4% | 94.5% | | | 62.4% | | |
| | (mass %) | Tetramer | 1.7% | 1.3% | 0.4% | | | 1.9% | | |
| | | By- | 8.1% | 6.7% | 5.0% | | | 35.4% | | |
| | | products | | | | | | | | |

| Propylene oligomer No. | | Oligomer | Oligomer | Oligomer | Oligomer | Oligomer | Oligomer | Oligomer | Oligomer |
|---|---|---|---|---|---|---|---|---|---|
| | | (1) | (2) | (3) | (4) | (5) | (6) | (7) | (8) |
| Compo- | Type V | 20.2% | 18.0% | 19.9% | 27.0% | 24.0% | 17.4% | 18.2% | 22.6% |
| sition | Type IV | 55.0% | 55.4% | 55.2% | 50.4% | 52.9% | 56.6% | 55.1% | 53.1% |
| of | Type III | 5.4% | 5.6% | 5.4% | 7.1% | 5.8% | 5.1% | 4.5% | 5.4% |
| propylene | Type II | 18.3% | 20.0% | 18.5% | 13.7% | 15.8% | 20.2% | 21.3% | 17.5% |
| oligomer | Type I | 1.1% | 0.9% | 1.0% | 1.8% | 1.5% | 0.7% | 0.9% | 1.4% |

It is found that the propylene oligomers obtained by the production methods in Examples 1 and 2 have a low Type V olefin concentration, and hence have a low degree of branching. In addition, the propylene oligomers can be obtained in high yield at a low temperature, making it possible to suppress degradation of the catalyst. Therefore, effects can be achieved such that the life of the catalyst is extended, and the frequency of maintenance is reduced. On the other hand, it is found that the propylene oligomers obtained in Comparative Examples 1 and 2 have a high Type V olefin concentration. Further, it is found that the propylene oligomers obtained in Comparative Examples 3 and 4 have a large amount of by-products and have a low selectivity. As apparent from the above, the propylene oligomers obtained by the production methods in Examples 1 and 2 are useful as a raw material for various olefin derivatives.

It is found that the propylene oligomer obtained by the production method in Example 3 has a low Type V olefin concentration, and hence has a low degree of branching, as compared to the propylene oligomer obtained in Comparative Example 5 in which the isomerization step is not performed. Further, it is found that a reduced amount of by-products are produced in the production method in Example 3. As apparent from the above, the propylene oligomer obtained by the production method in Example 3 is useful as a raw material for various olefin derivatives.

### Examples 4 to 6 and Comparative Examples 6 to 13

With respect to the below-mentioned zeolite catalysts, a BET specific surface area (total surface area) and a pore volume were measured using Autosorb-3, manufactured by Anton Paar GmbH.

In the BET analysis, the analysis soft attached to the apparatus was used. The BET specific surface area is a value determined by making a calculation from the slope and intercept of a line which is obtained by conducting a BET analysis at a relative pressure in the range of 0.005 to 0.1 using an adsorption isotherm obtained by the above measurement. A value of the nitrogen adsorption amount at a relative pressure of 0.95 in the adsorption isotherm was taken as a pore volume. Specifically, using two measured points around a relative pressure of 0.95, a nitrogen adsorption amount was calculated by an interpolation method.

A micropore surface area and a micropore volume were calculated from an analysis by a t-plot method using an adsorption isotherm obtained by the above measurement. First, in an analysis by a t-plot method, with respect to the adsorption isotherm under conditions that the average thickness (t) of the nitrogen which has adsorbed is in the range of 5 to 6.5 Å, linear approximation was conducted, and, from the slope of the resultant line, a specific surface area of pores other than micropores of the zeolite catalyst was calculated. Then, a difference between the above-obtained BET specific surface area and the specific surface area of pores other than micropores as obtained by a t-plot method was calculated and taken as a micropore specific surface area of the zeolite catalyst. The micropore volume was a value of the nitrogen adsorption amount at the y interception of the above-mentioned line obtained by approximation. For converting the relative pressure of adsorption isotherm to an average thickness (t) of the nitrogen which has adsorbed, the de Boer equation (source: J. H. de Boer, B. G. Linsen, Th. van der Plas, G. J. Zondervan, J. Catalysis, 4, 649 (1965)) was used.

From the obtained BET specific surface area and micropore surface area, a ratio of the micropore surface area to the total surface area was determined. Further, from the obtained pore volume and micropore volume, a ratio of the micropore volume to the pore volume was determined. The results are shown in Table 3.

Zeolite Catalyst A

MFI Type (another name: ZSM-5), 10-membered ring; HSZ-822HOD1A, manufactured by Tosoh Corp.; catalyst diameter: 1.5 mmø; catalyst length: 3 mm; extrusion molded article in a cylindrical shape)

Zeolite Catalyst B

BEA Type (another name: 13 zeolite), 12-membered ring; HSZ-930HOD1A, manufactured by Tosoh Corp.; catalyst diameter: 1.5 mmø; catalyst length: 3 mm; extrusion molded article in a cylindrical shape)

TABLE 3

| | | | | | BET | |
|---|---|---|---|---|---|---|
| | | | | | Specific | |
| | | | | | surface | |
| | | Micro- | | | area/ | |
| | BET | pore | | | Micropore | Pore |
| | Specific | specific | | Micro- | specific | volume/ |
| | surface | surface | Pore | pore | surface | Micro- |
| | area | area | volume | volume | area | pore |
| Catalyst | (m²/g) | (m²/g) | (cc/g) | (cc/g) | (a/b) | volume |
| Zeolite | 360 | 224 | 0.404 | 0.094 | 1.61 | 4.32 |
| catalyst A | | | | | | |
| Zeolite | 505 | 263 | 0.609 | 0.106 | 1.92 | 5.74 |
| catalyst B | | | | | | |

The composition of each of the propylene oligomers in the Examples and Comparative Examples was determined using a gas chromatography apparatus (6850 Network GC System, manufactured by Agilent Technologies, Inc.) as described below. As a column, DB-PETRO, manufactured by Agilent Technologies, Inc., (100 m×0.250 mm×0.50 μm) was used. As a carrier gas, helium was used, and the flow rate was 2.5 mL/minute. The injection temperature was 250° C., and the split ratio was 100. The reaction mixture was charged in the state of maintaining the oven temperature at 50° C., and maintained at 50° C. for 10 minutes. Then, the temperature of the oven was increased to 300° C. at a temperature increase rate of 3.13° C./minute, and the individual components were identified. A peak appearing at 8.0 to 11.8 minutes was ascribed to a propylene dimer, a peak appearing at 21.9 to 29.2 minutes was ascribed to a propylene trimer, a peak appearing at 36.7 to 43.9 minutes was ascribed to a propylene tetramer, a peak appearing at 43.9 minutes or more was ascribed to heavy compounds, such as propylene pentamer and polymers, and the other peaks were ascribed to by-products produced due to cracking. Areas of the peaks ascribed to the individual components were determined. The peak area ratio of each component was taken as a composition of each component in terms of a weight.

Further, among the peaks of the propylene tetramer, areas of the peaks appearing at 40.3 minutes and 40.7 minutes were determined in the same manner as mentioned above. A ratio of the areas of the peaks appearing at 40.3 minutes and 40.7 minutes to the total area of the peaks ascribed to the propylene tetramer was calculated, and determined as a 4,6,6-trimethyl-3-nonene concentration (% by mass) of the propylene tetramer.

### Example 4 (Production of Propylene Oligomer (9))

40 cc of zeolite A (MFI-type zeolite catalyst) and 40 cc of alumina balls (2 mmø; spherical; SSA-995, manufactured by Nikkato Corporation) were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, propylene was introduced so that the reaction pressure became 6.5 MPa and the feed rate became 60.6 cc/hour (LHSV=1.52 h⁻¹). For stabilizing the catalyst, a reaction was conducted for 70 days (1,668 hours), and then the reaction mixture was withdrawn to obtain a propylene oligomer (9). The average reaction temperature of the reaction tube was 131.9° C. Further, the propylene conversion was 70.8%.

The composition of the propylene oligomer (9) and the 4,6,6-trimethyl-3-nonene concentration of the propylene tetramer are shown in Table 4. In Table 4, “C6” means a propylene dimer, “C9” means a propylene trimer, “C12” means a propylene tetramer, “C15+” means heavy compounds, such as propylene pentamer and polymers, and “Crack” means by-products. Further, in Table 4, “Specific C12 concentration” means a 4,6,6-trimethyl-3-nonene concentration of the propylene tetramer.

Example 5 (Production of propylene oligomer (10)) 40 cc of the above-mentioned zeolite A and 40 cc of the alumina balls, which are the same as used in Example 4, were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, propylene was introduced so that the reaction pressure became 6.5 MPa and the feed rate became 59.8 cc/hour (LHSV=1.50 h⁻¹). For stabilizing the catalyst, a reaction was conducted for 63 days (1,500 hours), and then the reaction mixture was withdrawn to obtain a propylene oligomer (10). The average reaction temperature of the reaction tube was 132.2° C. Further, the propylene conversion was 79.1%.

The composition of the propylene oligomer (10) and the 4,6,6-trimethyl-3-nonene concentration of the propylene tetramer are shown in Table 4.

Example 6 (Production of propylene oligomer (11)) 40 cc of the above-mentioned zeolite A and 40 cc of the alumina balls, which are the same as used in Example 4, were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, propylene was introduced so that the reaction pressure became 6.5 MPa and the feed rate became 59.8 cc/hour (LHSV=1.50 If). For stabilizing the catalyst, a reaction was conducted for 41 days (972 hours), and then the reaction mixture was withdrawn to obtain a propylene oligomer (11). The average reaction temperature of the reaction tube was 151.9° C. Further, the propylene conversion was 93.7%.

The composition of the propylene oligomer (11) and the 4,6,6-trimethyl-3-nonene concentration of the propylene tetramer are shown in Table 4.

Comparative Example 6 (Production of propylene oligomer (12)) 40 cc of the above-mentioned zeolite B (BEA-type zeolite catalyst) and 40 cc of alumina balls (2 mmø; spherical; SSA-995, manufactured by Nikkato Corporation) were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, propylene was introduced so that the reaction pressure became 6.5 MPa and the feed rate became 63.5 cc/hour (LHSV=1.59 h⁻¹). For stabilizing the catalyst, a reaction was conducted for 102 days (2,436 hours), and then the reaction mixture was withdrawn to obtain a propylene oligomer (12). The average reaction temperature of the reaction tube was 117.8° C. Further, the propylene conversion was 46.0%.

The composition of the propylene oligomer (12) and the 4,6,6-trimethyl-3-nonene concentration of the propylene tetramer are shown in Table 4.

### Comparative Example 7 (Production of Propylene Oligomer (13))

40 cc of the above-mentioned zeolite B and 40 cc of the alumina balls, which are the same as used in Comparative Example 6, were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, propylene was introduced so that the reaction pressure became 6.5 MPa and the feed rate became 64.8 cc/hour (LHSV=1.62 h⁻¹). For stabilizing the catalyst, a reaction was conducted for 103 days (2,460 hours), and then the reaction mixture was withdrawn to obtain a propylene oligomer (13). The average reaction temperature of the reaction tube was 136.5° C. Further, the propylene conversion was 76.2%.

The composition of the propylene oligomer (13) and the 4,6,6-trimethyl-3-nonene concentration of the propylene tetramer are shown in Table 4.

### Comparative Example 8 (Production of Propylene Oligomer (14))

40 cc of the above-mentioned zeolite B and 40 cc of the alumina balls, which are the same as used in Comparative Example 6, were mixed with each other, and a fixed bed reaction tube made of stainless steel was filled with the resultant mixture.

The inside of the reaction tube was treated in a nitrogen gas flow at 200° C. for 3 hours, and cooled to 25° C.

Then, propylene was introduced so that the reaction pressure became 6.5 MPa and the feed rate became 62.9 cc/hour (LHSV=1.57 h⁻¹). For stabilizing the catalyst, a reaction was conducted for 99 days (2,364 hours), and then the reaction mixture was withdrawn to obtain a propylene oligomer (14). The average reaction temperature of the reaction tube was 153.1° C. Further, the propylene conversion was 91.6%.

The composition of the propylene oligomer (14) and the 4,6,6-trimethyl-3-nonene concentration of the propylene tetramer are shown in Table 4.

Comparative Example 9 (Production of propylene oligomer (15))

A fixed bed reaction tube made of stainless steel was filled with 20 cc of the solid phosphoric acid catalyst obtained in Production Example 1.

Then, propylene was introduced so that the reaction pressure became 6.5 MPa and the feed rate became 30 cc/hour (LHSV=1.50 h⁻¹). For preventing the solid phosphoric acid catalyst from lowering in the activity, water was simultaneously introduced in an amount of 30.7 ppm by mass, based on the mass of the raw material. A reaction was conducted for 18 days (432 hours), and then the reaction mixture was withdrawn to obtain a propylene oligomer (15). The average reaction temperature was 167.0° C. Further, the propylene conversion was 49.5%.

The composition of the propylene oligomer (15) and the 4,6,6-trimethyl-3-nonene concentration of the propylene tetramer are shown in Table 4.

### Comparative Example 10 (Production of Propylene Oligomer (16))

A fixed bed reaction tube made of stainless steel was filled with 10 cc of the solid phosphoric acid catalyst obtained in Production Example 1.

Then, propylene was introduced so that the reaction pressure became 6.5 MPa and the feed rate became 44.4 cc/hour (LHSV=4.44 h⁻¹). For preventing the solid phosphoric acid catalyst from lowering in the activity, water was simultaneously introduced in an amount of 84 ppm by mass, based on the mass of the raw material. A reaction was conducted for 4 days (96 hours), and then the reaction mixture was withdrawn to obtain a propylene oligomer (16). The average reaction temperature was 189.5° C. Further, the propylene conversion was 76.3%.

The composition of the propylene oligomer (16) and the 4,6,6-trimethyl-3-nonene concentration of the propylene tetramer are shown in Table 4.

### Comparative Example 11 (Production of Propylene Oligomer (17))

A fixed bed reaction tube made of stainless steel was filled with 20 cc of the solid phosphoric acid catalyst obtained in Production Example 1.

Then, propylene was introduced so that the reaction pressure became 6.5 MPa and the feed rate became 31.1 cc/hour (LHSV=1.55 h⁻¹). For preventing the solid phosphoric acid catalyst from lowering in the activity, water was simultaneously introduced in an amount of 54.3 ppm by mass, based on the mass of the raw material. A reaction was conducted for 10 days (240 hours), and then the reaction mixture was withdrawn to obtain a propylene oligomer (17). The average reaction temperature was 167.8° C. Further, the propylene conversion was 83.9%.

The composition of the propylene oligomer (17) and the 4,6,6-trimethyl-3-nonene concentration of the propylene tetramer are shown in Table 4.

### Comparative Example 12 (Production of Propylene Oligomer (18))

A fixed bed reaction tube made of stainless steel was filled with 60 cc of the solid phosphoric acid catalyst obtained in Production Example 1.

Then, propylene was introduced so that the reaction pressure became 6.5 MPa and the feed rate became 31.7 cc/hour (LHSV=0.53 h⁻¹). For preventing the solid phosphoric acid catalyst from lowering in the activity, water was simultaneously introduced in an amount of 16.7 ppm by mass, based on the mass of the raw material. A reaction was conducted for 15 days (360 hours), and then the reaction mixture was withdrawn to obtain a propylene oligomer (18). The average reaction temperature was 129.0° C. Further, the propylene conversion was 80.0%.

The composition of the propylene oligomer (18) and the 4,6,6-trimethyl-3-nonene concentration of the propylene tetramer are shown in Table 4.

### Comparative Example 13 (Production of Propylene Oligomer (19))

A fixed bed reaction tube made of stainless steel was filled with 20 cc of the solid phosphoric acid catalyst obtained in Production Example 1.

Then, propylene was introduced so that the reaction pressure became 6.5 MPa and the feed rate became 29.2 cc/hour (LHSV=1.46 If). For preventing the solid phosphoric acid catalyst from lowering in the activity, water was simultaneously introduced in an amount of 55.7 ppm by mass, based on the mass of the raw material. A reaction was conducted for 38 days (912 hours), and then the reaction mixture was withdrawn to obtain a propylene oligomer (19). The average reaction temperature was 185.7° C. Further, the propylene conversion was 88.0%.

The composition of the propylene oligomer (19) and the 4,6,6-trimethyl-3-nonene concentration of the propylene tetramer are shown in Table 4.

TABLE 4

| | | Reaction | | | | | | | Specific C12 |
|---|---|---|---|---|---|---|---|---|---|
| | | temperature | Conversion | Composition (%) | | | | | concentration |
| | Catalyst | (° C.) | (%) | C6 | C9 | C12 | C15+ | Crack | (Mass %) |
| Example 4 | Zeolite A | 131.9 | 70.8 | 81.2 | 14.5 | 4.0 | 0.0 | 0.3 | 59.6 |
| Example 5 | Zeolite A | 132.2 | 79.1 | 74.8 | 18.2 | 6.7 | 0.0 | 0.3 | 59.9 |
| Example 6 | Zeolite A | 151.9 | 93.7 | 36.6 | 36.9 | 23.3 | 1.5 | 1.7 | 40.1 |
| Comparative | Zeolite B | 117.8 | 46.0 | 14.6 | 65.6 | 14.4 | 2.5 | 3.0 | 17.5 |
| Example 6 | | | | | | | | | |
| Comparative | Zeolite B | 136.5 | 76.2 | 6.2 | 55.3 | 23.5 | 6.7 | 8.4 | 15.4 |
| Example 7 | | | | | | | | | |
| Comparative | Zeolite B | 153.1 | 91.6 | 4.0 | 35.5 | 26.3 | 17.7 | 16.6 | 5.9 |
| Example 8 | | | | | | | | | |
| Comparative | Solid | 167.0 | 49.5 | 10.4 | 71.5 | 12.9 | 0.0 | 5.2 | 6.9 |
| Example 9 | phosphoric | | | | | | | | |
| | acid | | | | | | | | |
| Comparative | Solid | 189.5 | 76.3 | 7.0 | 69.3 | 15.3 | 0.7 | 7.7 | 4.5 |
| Example 10 | phosphoric | | | | | | | | |
| | acid | | | | | | | | |
| Comparative | Solid | 167.8 | 83.9 | 3.4 | 62.8 | 24.7 | 0.9 | 8.2 | 6.4 |
| Example 11 | phosphoric | | | | | | | | |
| | acid | | | | | | | | |
| Comparative | Solid | 129.0 | 80.0 | 6.7 | 76.6 | 12.4 | 1.2 | 3.1 | 13.2 |
| Example 12 | phosphoric | | | | | | | | |
| | acid | | | | | | | | |
| Comparative | Solid | 185.7 | 88.0 | 3.6 | 59.1 | 23.6 | 1.8 | 11.9 | 5.1 |
| Example 13 | phosphoric | | | | | | | | |
| | acid | | | | | | | | |

As seen from Table 3, the zeolite catalyst A used in the production method in the Examples had a BET specific surface area smaller than that of the zeolite catalyst B used in the production method in Comparative Examples 6 to 8, but had a relatively large micropore specific surface area, and accordingly had a small ratio of the BET specific surface area to the micropore specific surface area (a/b).

It is found that the propylene oligomers in the Examples, which were produced using the zeolite catalyst (zeolite A) in which a/b is 1.61, have a high 4,6,6-trimethyl-3-nonene concentration of the propylene tetramer (C12). On the other hand, the propylene oligomers in Comparative Examples 6 to 8, which were produced using the zeolite catalyst (zeolite B) in which a/b is 1.92, had a low 4,6,6-trimethyl-3-nonene concentration of the propylene tetramer (C12). Further, the propylene oligomers in Comparative Examples 9 to 13, which were produced using the solid phosphoric acid catalyst, had a low 4,6,6-trimethyl-3-nonene concentration of the propylene tetramer (C12). From the above results, it was found that the ratio of the BET specific surface area to the micropore specific surface area (a/b) in the zeolite catalyst affects production of 4,6,6-trimethyl-3-nonene.

With respect to the composition, the propylene oligomers in Examples 4 to 6 had a relatively high proportion of the propylene dimer (C6). On the other hand, the propylene oligomers in Comparative Examples 6 to 8 and 9 to 13 had a low proportion of the propylene dimer (C6), and had a relatively high proportion of the propylene trimer (C9). From the above results, it is presumed that, in the production method in the Examples, a route of reaction different from that in the production method in the Comparative Examples, namely, a reaction route of dimerization of propylene dimers selectively proceeded. With respect to Examples 4 to 6, when the propylene dimer (C6) is recycled, it can be expected that a dimerization reaction of the propylene dimers selectively proceeds, making it possible to increase the proportion of the propylene tetramer (C12) and the 4,6,6-trimethyl-3-nonene concentration.

1. A method for producing a propylene oligomer, comprising:
oligomerizing propylene at lower than 160° C. in the presence of at least one member selected from a group consisting of a catalyst comprising crystalline molecular sieve and a catalyst comprising phosphoric acid,
fractional distilling to obtain a fraction comprising a propylene trimer, a propylene tetramer, or a mixture thereof, and
isomerizing the propylene trimer, the propylene tetramer, or the mixture thereof contained in the fraction in the presence of a catalyst comprising phosphoric acid.

2. The method for producing a propylene oligomer according to claim 1, wherein the crystalline molecular sieve is at least one member selected from a group consisting of a 10-membered ring zeolite and a 12-membered ring zeolite.

3. The method for producing a propylene oligomer according to claim 1, wherein the crystalline molecular sieve is MFI-type zeolite.

4. The method for producing a propylene oligomer according to claim 1, wherein the catalyst comprising phosphoric acid used in the isomerization step is a solid phosphoric acid catalyst.

5. The method for producing a propylene oligomer according to claim 1, wherein the catalyst containing comprising phosphoric acid used in the oligomerization step is a solid phosphoric acid catalyst.

6. The method for producing a propylene oligomer according to claim 1, wherein the isomerization step is conducted at 160° C. or higher.

7. A method for producing a propylene oligomer, comprising:
isomerizing an oligomer comprising a propylene trimer, a propylene tetramer, or a mixture thereof at less than the critical pressure of propylene in the presence of at least one member selected from a group consisting of a catalyst comprising phosphoric acid.

8. The method for producing a propylene oligomer according to claim 7, wherein the catalyst is a solid phosphoric acid catalyst.

9. The method for producing a propylene oligomer according to claim 7, wherein the isomerizing the oligomer is conducted at a pressure of 3.00 MPa or less, in terms of a gauge pressure.

10. A propylene oligomer, comprising a propylene tetramer having a 4,6,6-trimethyl-3-nonene concentration of 30% by mass or more.

11. A method for producing a propylene oligomer, comprising:
oligomerizing propylene in the presence of a catalyst comprising a crystalline molecular sieve,
wherein when a BET specific surface area of the crystalline molecular sieve, as obtained by a nitrogen adsorption method, is taken as “a” [m²/g], and a micropore specific surface area of the crystalline molecular sieve, as obtained by subjecting an adsorption isotherm measured by a nitrogen adsorption method to analysis in accordance with a t-plot method, is taken as “b” [m²/g], a/b is 1.8 or less.

12. The method for producing a propylene oligomer according to claim 11, wherein the oligomerizing propylene produces a propylene oligomer comprising a propylene tetramer having a 4,6,6-trimethyl-3-nonene concentration of 30% by mass or more.

13. The method for producing a propylene oligomer according to claim 11, wherein the crystalline molecular sieve is a 10-membered ring zeolite.

14. The method for producing a propylene oligomer according to claim 11, wherein the crystalline molecular sieve is MFI-type zeolite.

15. The method for producing a propylene oligomer according to claim 11, wherein a reaction temperature of the oligomerizing propylene is lower than 220° C.
